SEQUENCE LISTING (2 sequences)

SEQ ID NO: 1 - protein, 19 aa, Homo sapiens
CAIIRVLTGTEKKTGVYMQ

SEQ ID NO: 2 - protein, 140 aa, Homo sapiens
MDGKGSYQLKGWKLTQQQFVALLWKRLLIARRSRKGFFAQIVLPAVFVCIALVFSLIVPPFGKYPSLELQPWMYNEQYTFVSNDAPEDMGTQELLNALTKDPGFGTRCMEGNPIPDTPCLAGEEDWTISPVPQSIVDLFQ

(19) United States
(12) Patent Application Publication
(10) Pub. No.: US 2007/0178086 A1
(43) Pub. Date: Aug. 2, 2007

(54) Monoclonal antibody against ABCA1

(75) Inventors: Dmitri Sviridov, Brighton East (AU); Nigora Mukhamedova, Brighton East (AU); Ying Fu, Watsonia (AU)

Correspondence Address: BIRCH STEWART KOLASCH & BIRCH, PO BOX 747, FALLS CHURCH, VA, 22040-0747, US

(73) Assignee: Baker Medical Research Institute, Prahran (AU)

(21) Appl. No.: 11/604,267

(22) Filed: Nov. 27, 2006

Related U.S. Application Data
(63) Continuation-in-part of application No. PCT/AU05/00753, filed on May 27, 2005.

(30) Foreign Application Priority Data
May 27, 2004 (AU) 2004902842

(51) Int. Cl. A61K 39/395; G01N 33/53; C12N 5/06; C07K 16/28

(52) U.S. Cl. 424/143.1; 435/7.1; 530/350; 530/388.22; 435/334

(57) ABSTRACT

The present invention provides a binding domain of ABCA1, wherein binding of a ligand to the domain is capable of modulating the biological activity of ABCA1. Also provided are methods for screening compounds capable of modulating ABCA1 activity, and antibodies useful in modulating ABCA1 activity.

### RELATED APPLICATIONS

This application is a Continuation-In-Part of copending PCT International Application No. PCT/AU2005/000753 filed on May 27, 2005, which designated the United States, and on which priority is claimed under 35 U.S.C. § 120. This application also claims priority under 35 U.S.C. § 119(a) on Patent Application No(s). 2004902842 filed in Australia on May 27, 2004. The entire contents of each of the above documents is hereby incorporated by reference.

### FIELD

The present invention relates to the field of the control of blood lipids. More particularly, the present invention relates to compositions and methods for modulating the metabolism and transport of lipids in the blood.

### BACKGROUND

Cholesterol is essential for the growth and viability of higher organisms. It is a lipid that modulates the fluidity of eukaryotic membranes, and is the precursor to steroid hormones such as progesterone, testosterone, and the like. Cholesterol can be obtained from the diet, or synthesized internally in the liver and the intestines. Cholesterol is transported in body fluids to specific targets by lipoproteins, which are classified according to increasing density. For example, low density lipoprotein cholesterol (LDL) is responsible for transport of cholesterol to and from the liver and to peripheral tissue cells, where LDL receptors bind LDL, and mediate its entry into the cell.

Although cholesterol is essential to many biological processes in mammals, elevated serum levels of LDL cholesterol are undesirable, in that they are known to contribute to the formation of atherosclerotic plaques in arteries throughout the body, which may lead, for example, to the development of coronary artery diseases. Conversely, elevated levels of high density lipoprotein cholesterol (HDL-C) have been found, based upon human clinical data, and animal model systems, to protect against development of coronary diseases.

In general, excess cholesterol is removed from the body by a pathway involving high density lipoproteins (HDLs). Cholesterol is “effluxed” from cells by one of two processes—either by passive transfer to mature HDL, or an active transfer to apolipoprotein A-1. The latter process is mediated by a protein known as ATP binding cassette transporter 1 (ABCA1, or alternatively referenced as ABC-1). In the latter process, lipid-poor HDL precursors acquire phospholipid and cholesterol, which leads to increased plasma levels of mature HDL particles. HDL cholesterol is eventually transported to the liver in a process known as “reverse cholesterol transport”, where it is either recycled or excreted as bile.

ABCA1 knockout mice do not have HDL and are susceptible for atherosclerosis. Overexpression of ABCA1 results in higher plasma HDL levels and enhanced protection against development of atherosclerosis. Lack of ABCA1 in humans is the cause of Tangier disease, a disorder characterized by absence of HDL in plasma and non-existing reverse cholesterol transport. Mutations of ABCA1 in humans is a predominant causes of hypoalphalipoproteinaemia. Staggering progress in studying structure and function of ABCA was however slowed by lack of monoclonal antibody against ABCA1. Apart from a leading sequence of 60 amino acids absent in the mouse ABCA1 there is 95% match between sequences of human and mouse ABCA1.

One method of treatment aimed at reducing the risk of formation of atherosclerotic plaques in arteries relates to decreasing plasma lipid levels. Such a method includes diet changes, and/or treatment with drugs such as derivatives of fibric acid (clofibrate, gemfibrozil, and fenofibrate), nicotinic acid, and HMG-CoA reductase inhibitors, such as mevinolin, mevastatin, pravastatin, simvastatin, fluvastatin, and lovastatin, which reduce plasma LDL cholesterol levels by either inhibiting the intracellular synthesis of cholesterol or inhibiting the uptake via LDL receptors. In addition, bile acid-binding resins, such as cholestyrine, colestipol and probucol decrease the level of LDL-cholesterol by reducing intestinal uptake and increasing the catabolism of LDL-cholesterol in the liver.

It is desired to provide alternative therapies aimed at reducing the risk of formation of atherosclerotic plaques in arteries, especially in individuals deficient in the removal of cholesterol from artery walls via the HDL pathway. Given that HDL levels are generally related to the expression of ABCA-1, one method of increasing HDL levels would be to increase the expression of ABCA-1. Accordingly, it is desired to provide methods and agents that modulate the biological activity of ABCA-1 in mammals, thus increasing cholesterol efflux and raising HDL cholesterol levels in blood. This would be useful inter alia for the treatment of various disease states characterized by low HDL levels, in particular coronary artery disease.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed in Australia before the priority date of this application.

### BRIEF DESCRIPTION OF THE FIGURES

FIG. 1 shows a Western blot using antibodies against ABCA1. Cells were lysed in RIPA buffer and proteins were separated on a 7.5% SDS-polyacrylamide gel followed by immunoblotting. 1—THP-1 cells; 2—HEK2931 cells transfected with mouse ABCA1; 3—HepG2 cells; 4—CHOP cells.

FIG. 2. shows confocal microscopy of THP-1 cells with monoclonal antibodies. THP-1 cells were grown on sterile plastic cover slips to approximately 60% confluence. Cells were fixed in acetone for 20 min, washed with PBS and incubated for 1 h with the antibodies. Cells were then washed again and incubated in the dark for 1 h with Texas Red labeled anti mouse IgG.

FIG. 3. shows the effect of monoclonal antibodies on cholesterol efflux from THP-1 cells. Cholesterol efflux experiments were conducted as described in the Examples section. Cholesterol efflux is expressed as the percentage of labeled cholesterol moved from cells to medium (i.e. radioactivity in the medium/radioactivity in the medium+radioactivity in the cells). Means±SD of quadruplicate determinations are shown. *p<0.01 versus non-specific IgM.

FIG. 4. shows the effect of monoclonal antibodies on cholesterol efflux from THP-1 cells. Cholesterol efflux experiments were conducted as described in the Examples section. Cholesterol efflux is expressed as the percentage of labeled cholesterol moved from cells to medium (i.e. radioactivity in the medium/radioactivity in the medium+radioactivity in the cells). Means±SD of quadruplicate determinations are shown. *p<0.01 versus non-specific IgM.

FIG. 5 shows Western blotting of activated RAW264.7 cells using antibody against ABCA1. RAW 264.7 cells were activated by incubation for 18 h with 1 μmol/L of TO901317. Cells were then lysed in RIPA buffer and proteins were separated on a 7.5% SDS-polyacrylamide gel followed by immunoblotting and staining with antibody NDF 4C2.

FIG. 6 shows that antibody could detect changes in ABCA1 abundance. RAW 264.7 mouse macrophage were activated with LXR activator TO901317. The same amount of cell protein from activated and non-activated cells was analyzed by Western blot using antibody NDF4C2 followed by densitometry. Seven-fold increase in abundance of ABCA1 in RAW 264.7 cells was detected.

FIG. 7 shows a schematic representation of ABCA1 and location of the antibody epitopes. Closed circles denote fragments of ABCA1 chosen as targets for the development of the antibodies. Shaded circles denote PEST domain.

FIG. 8. shows Western blotting of activated or non-activated RAW 264.7 cells (A) or THP-1, HEK 293/hABCA1, 3T3 and RAW 264.7 cells (B) using antibodies against ABCA1. Panel A —RAW 264.7 cells were activated or not by incubation for 18 h with 0.3 mmol/L of cAMP. Cells were then lysed as described in Materials and Methods and proteins were separated on a 6% SDS-PAGE followed by immunoblotting with the indicated antibodies. Panel B—THP-1 cells (differentiated with PMA), HEK 293 cells transiently transfected with human ABCA1, 3T3 cells or RAW 264.7 cells were grown to confluency. Cells were then lysed as described in Example 1 and proteins were separated on a 6% SDS-polyacrylamide gel followed by immunoblotting with NDF4C2 antibody.

FIG. 9. shows confocal microscopy of THP-1 cells stained with antibodies against ABCA1.

THP-1 cells were grown on sterile cover slips to approximately 60% confluence, differentiated by incubation for 48 h with 100 ng/ml of PMA, and ABCA1 expression was activated by incubation for 18 h with 4 μmol/L of TO-901317. Cells were fixed with 4% paraformaldehyde, permeabilized with 0.2% Triton X-100, washed and stained. Panel A—primary antibody —NDF4C2, secondary antibody—Alexa Fluor 488 goat anti-mouse IgM; Panels B, E, H—primary antibody—polyclonal anti-calnexin, secondary antibody—Texas Red goal anti-rabbit antibody; Panel C— overlap of A and B; Panel D—primary antibody —NDF6F1, secondary antibody—Alexa Fluor 488 goat anti-mouse IgM; Panel F—overlap of D and E; Panel G—primary antibody —NDF3F9, secondary antibody—Alexa Fluor 488 goat anti-mouse IgG; Panel I—overlap of G and H; Panel J—primary antibody—polyclonal anti-ABCA1 antibody, secondary antibody—Texas Red goal anti-rabbit antibody; Panel K—primary antibody monoclonal anti-calnexin antibody secondary antibody—Alexa Fluor 488 goat anti-mouse IgG; Panel L—overlap of J and K. Bar—10 μm.

FIG. 10. shows the effect of the monoclonal antibodies on cholesterol (Panel A) and phospholipid (Panel B) efflux from THP-1 cells. THP-1 cells were grown in 12-well plates and ABCA1 expression was boosted by incubation with 4 μmol/L of LXR agonist TO-901317. Cellular cholesterol was labeled by incubation of cells in serum-containing medium with [³H]-cholesterol or [¹⁴C] choline for 48 h in a CO₂ incubator. Cells were then washed and incubated for 18 h at 37° C. with serum-free medium containing indicated antibodies at the final concentration 20 μg/ml. Cells were washed and incubated for 2 h at 37⁰ C with serum-free medium containing 30 μg/ml apoA-I. The medium was then collected, centrifuged for 15 min at 4° C. at 10,000×g and aliquots of supernatant and cells were either counted in a β-counter (A) or separated on TLC and bands corresponding to phospholipid were counted (B). The efflux was expressed as a proportion of [³H]cholesterol or [¹⁴C] phospholipid transferred from cells to medium. Mean±SEM of quadruplicate determinations are shown. *p<0.01 versus no antibody.

FIG. 11. shows the effect of the antibody NDF6F1 on cholesterol efflux from THP-1 cells activated with TO-901317 (Panel A) or non-activated (Panel B). THP-1 cells were grown in 12-well plates and when indicated ABCA1 expression was boosted (A) or not (B) by incubation with 4 μmol/L of LXR agonist TO-901317. Cellular cholesterol was labeled by incubation of cells in serum-containing medium with [³H]-cholesterol for 48 h. Cells were then washed and incubated for 18 h at 37° C. with serum-free medium containing indicated concentration of NDF6F1 antibody. Cells were washed and incubated for 3 h at 37⁰ C with serum-free medium containing 30 μg/ml apoA-I. The medium was then collected, centrifuged for 15 min at 4° C. at 10,000×g and aliquots of supernatant were counted in a β-counter. Cells were harvested and radioactivity counted. Cholesterol efflux was expressed as a proportion of [³H]cholesterol transferred from cells to medium. Mean±SEM of quadruplicate determinations are shown. *p<0.01 versus no antibody.

FIG. 12. shows the effect of the monoclonal antibodies on ¹²⁵I-apoA-I binding (A) and internalization (B) by THP-1 cells. THP-1 cells were grown in 12-well plates and when indicated the expression of ABCA1 was boosted by incubation with 4 μmol/L of LXR agonist TO-901317. Cells were incubated for 2 h at 37° C. in serum-free medium containing 0.1% BSA (essentially fatty-acid free, Sigma), 5 μg/ml of ¹²⁵I-apoA-I in the presence or absence of 250 μg/ml of unlabeled apoA-I. Cells were then washed 3 times with serum-free medium containing 0.1% BSA and once with Ca⁺⁺, Mg⁺⁺ free PBS and treated with 0.05% trypsin/0.002% EDTA. The reaction was stopped by adding 100 μl of fetal calf serum. Radioactivity in the medium (binding) and cells (internalization) was determined in a γ-counter. *p<0.01 versus no antibody.

FIG. 13 shows the effect of apoA-I and the antibody NDF6F1 on ABCA1 stability (A) and cell surface abundance (B). Panel A—ABCA1 expression in THP-1 cells was boosted by incubation for 18 h with LXR agonist TO-901317 (4 μmol/L). Cells were then washed and incubated for 18 h in the serum-free medium alone or medium containing lipid free apoA-I (30 μg/ml) or antibody NDF6F1 (5 μg/ml) or co-incubated with the hybridoma cells producing the NDF6F1 antibody. Suspension hybridoma cells were removed from flask after the incubation. THP-1 cells were then lysed as described in Materials and Methods and proteins were separated on a 6% SDS-PAGE followed by immunoblotting with NDF4C2 antibody or anti-β-actin monoclonal antibody. B—THP-1 cells were incubated for 24 h with TO-901317 (4 μmol/L), washed and incubated for 18 h in the serum-free medium, medium containing TO-901317 (4 μmol/L), antibodies NDF6F1 or NDF4C2 (10 μg/ml) or apoA-I (50 μg/ml). Cells were then biotinylated, biotinylated proteins precipitated and analyzed by Western Blot as described in “Materials and Methods”.

FIG. 14. shows the effect of apoA-I and the antibodies NDF6F1 and NDF4C2 on the rate of ABCA1 degradation. THP-1 cells were differentiated and ABCA1 expression was boosted by incubation with TO-901317 (4 μmol/L) for 18 h. Cells were then washed and incubated for the indicated periods of time with serum-free medium (control), fresh TO-901317 (4 μmol/L), apoA-I (50 μg/ml) or NDF6F1 or NDF4C2 antibodies (10 μg/ml). Cells were then washed, fixed with 3.7% formaldehyde in PBS for 20 min and permeabilized with three 10-min washes with 0.1% Triton X-100 in PBS. After blocking cells were incubated overnight with monoclonal NDF4C2 anti-ABCA1 antibodies and rabbit polyclonal anti-β-actin antibodies. Secondary detection was carried out using two species-specific infrared fluorescent dye conjugated antibodies. After 1 h incubation targets were simultaneously visualized and quantitated using the Odyssey Infrared Imaging Scanner (Li-Cor, Uncoln, NB). ABCA1 abundance was presented relative to β-actin abundance.

FIG. 15 shows cholesterol efflux from HeLa cells mock-transfected (A) or transfected with ABCA1 (B) or ΔPEST-ABCA1 (C). HeLa cells were transfected with pCMV-β-gal plasmid (A), ABCA1 (B) or ΔPEST-ABCA1 (C) as described in “Materials and Methods.” Cellular cholesterol was labeled by incubating the cells in serum-containing medium with [³H]-cholesterol for 48 h. Cells were then washed and incubated for 18 h at 37° C. in serum-free medium in the presence or absence of the antibodies (final concentration 10 μg/mL). Cells were washed and incubated for 2 h at 37° C. in serum-free medium containing 30 μg/ml apoA-I. The medium was then collected, centrifuged for 15 min at 4° C. at 10,000×g and aliquots of supernatant were counted in a β-counter. Cells were harvested and radioactivity was counted. Cholesterol efflux was expressed as a proportion of [³H]cholesterol transferred from cells to medium. Mean±SEM of quadruplicate determinations are shown. *p<0.01 versus no antibody.

FIG. 16. shows the effect of the antibodies on cholesterol (A) and cholesteryl ester (B) synthesis in macrophages. RAW 264.7 macrophages were preincubated with the antibodies (final concentration 5 μg/ml) for 18 h at 37° C. Cells were then incubated for 2 h at 37° C. with [³H] acetate and [¹⁴X]oleic acid (complexed to BSA). Cells were washed and lipids were extracted and analyzed by TLC. Spots of cholesterol and cholesteryl oleate were identified by standards (Sigma), scraped and counted in a β-counter. *p<0.01 versus no antibody.

### SUMMARY OF THE INVENTION

The present invention provides a binding domain of ABCA1, wherein binding of a ligand to the domain is capable of modulating a biological activity of ABCA1. Applicants have identified two binding domains on the ABCA1 protein that are involved in modulating a biological activity of the molecule. Without wishing to be limited by theory, Applicants propose that binding of ligands to one or both of these domains may enhance the stability of the ABCA1 molecule. It is further proposed that binding of ligands may prevent degradation of ABCA1, which is the main mechanism of regulation of ABCA1 concentration in the cell. Applicants further propose that localization of the ABCA1 molecule within the cell is affected by binding of ligands to the domains. Other biological activities such as the binding of ABCA1 to apoA-I, and internalization of ABCA1 may also be affected by binding of ligands to the domains. In addition, the binding domains are proposed to be involved in the ability of ABCA1 to efflux cholesterol or phospholipid.

One of the binding domains includes or consists of the extracellular loop connecting the first and second transmembrane domains of ABCA1. Preferably, the binding domain includes or consists of amino acid residues from the human ABCA1 region from about position 602 to about position 620, or a functional equivalent or fragment thereof. In another preferred form of the invention the binding domain includes or consists of amino acid residues from the mouse ABCA1 region from position 542 to about position 560. In a highly preferred form of the invention the sequence is:

(SEQ ID NO:1)

CAIIRVLTGTEKKTGVYMQ

or a functional equivalent or fragment thereof.

The second binding domain includes or consists of a part of the extracellular loop connecting the 7^{th} and 8^{th} transmembrane domains of ABCA1. In a preferred form of the invention the binding domain includes or consists of amino acid residues from the mouse ABCA1 region from about position 1251 to about position 1390 or a functional equivalent or fragment thereof. Preferably, the binding domain includes or consists of amino acid residues from the human ABCA1 region from about position 1311 to about position 1450 or a functional equivalent or fragment thereof. In a highly preferred form of the invention the sequences is:

(SEQ ID NO:2)

MDGKGSYQLKGWKLTQQQFVALLWKRLLIARRSRKGFFAQIVLPAVFVCI
ALVFSLIVPPFGKYPSLELQPWMYNEQYTFVSNDAPEDMGTQELLNALTK
DPGFGTRCME GNPIPDTPCLAGEEDWTISPVPQSIVDLFQ.

or a functional equivalent or fragment thereof.

In another aspect the present invention provides a method of identifying a ABCA1 binding domain agonist or antagonist the method including exposing a potential agonist or antagonist to a ABCA1 binding domain or portion thereof, and determining the ability of the potential agonist or antagonist to bind to the ABCA1 binding domain or otherwise interfere with the binding of the ABCA1 binding domain with an associated molecule.

In another aspect the present invention also provides a method for treating or preventing a disease related to a biological activity of ABCA1, the method including administering to a subject an effective amount of an ABCA 1 agonist or antagonist as described herein. The disease related to a biological activity of ABCA1 may be a disease related to cholesterol or phospholipid efflux. Preferably, the disease is selected from the group including but not limited to Tangier disease, coronary heart disease, atherosclerosis, and acquired immune deficiency syndrome.

The present invention also provides compositions including the ABCA1 agonists and antagonists described herein, in combination with a carrier. Those skilled in the art will be able by routine experimentation to determine appropriate buffers, stabilisers, preservatives, excipients, adjuvants, solvents and the like suitable for preparing such a composition. The composition may further include other therapeutic compounds in addition to those defined above.

Applicants have identified a number of antibodies capable of modulating the biological activity or localization of ABCA1. Accordingly, another aspect the present invention provides an antibody selected from the group consisting of NDF4C2, NDF3F9, NDF2D12, or NDF6F1 or a functional equivalent or fragment thereof. Importantly, it has been found that these antibodies are able to bind to a binding domain of ABCA1, thereby modulating the biological activity or localization.

In another aspect the present invention provides a method of isolating a subpopulation of cells from a population of cells, the method including exposing the population of cells to an antibody described herein, and separating the cells that specifically bind to the antibody from those that do not specifically bind to the antibody. The present invention also provides a subpopulation of cells isolated using the method.

### DETAILED DESCRIPTION OF THE INVENTION

In one aspect the present invention provides a binding domain of ABCA1, wherein binding of a ligand to the domain is capable of modulating a biological activity of ABCA1. Applicants have identified two binding domains on the ABCA1 protein that are involved in modulating various biological activities of the molecule.

In one form of the binding domain, the biological activity is selected from the group consisting of: apoA-binding, internalization, localization, stability, ability to efflux cholesterol, and ability to efflux phospholipid. The natural acceptor of lipids (cholesterol and/or phospholipid) released from cells via an ABCA1 dependent mechanism is apoA-I, and it is suggested that binding of apoA-I to ABCA1 is required for loading lipids to apoA-I, which occurs on the plasma membrane. Internalization of the ABCA1-apoA-I complex is required for lipid loading of apoA-I to occur intracellularly. It is also proposed that the presence of ABCA1 on the plasma membrane leads to a redistribution of lipids making them available for efflux. ApoA-I in addition to being a cholesterol acceptor is essential for the regulation of ABCA1 abundance by preventing its degradation. By using a panel of three monoclonal antibodies against ABCA1 Applicants propose that distinct sites of ABCA1 may be responsible for apoA-I binding, internalization, ability to support cholesterol efflux and stability of ABCA1.

As discussed throughout this application monoclonal antibodies have been raised against two binding domain regions to further investigate structure-function relationships of ABCA1. However, it must be emphasised, that irrespective of the mechanisms by which the ABCA1 binding domains interact with the various antibodies, ligands, agonists and antagonists described herein, the biological result is an alteration in the efflux of cholesterol or phospholipid. A further biological result is alteration in the levels of HDL in the serum (further discussed infra). Importantly, efflux of cholesterol and phospholipid, and alteration in the level of HDL are proposed to be useful tools in the prevention and treatment of a number of important diseases such as atheroscleosis.

An important finding is that while two large extracellular loops of ABCA1 were confirmed to be critical for the interaction with apoA-I and cholesterol efflux, the events following apoA-I binding were different for each loop and independent of each other. The first extracellular loop is proposed to be involved in apoA-I binding for the purpose of cholesterol efflux or for internalization of apoA-I/ABCA1 complex. Even within the loop, the functions of apoA-I binding and cholesterol efflux are likely to be dissociated. The binding of apoA-I to the fourth extracellular loop is most likely required for another apoA-I function—preventing ABCA1 degradation.

The term “ABCA1” as used herein is intended to include all members of the ABCA1 family, including but not limited to that disclosed in Genbank entries having the following accession details:

| Accession | Species |
|---|---|
| gi495256 | Mus musculus |
| gi26342297 | Mus musculus |
| gi34577068 | Mus musculus |
| gi28912191 | Rattus norvegicus |
| gi31342527 | Rattus norvegicus |
| gi5734100 | Homo sapiens |
| gi21536375 | Homo sapiens |
| gi15212106 | Homo sapiens |
| gi4128032 | Homo sapiens |
| gi9755158 | Homo sapiens |
| gi18028982 | Gallus gallus |

In one form of the invention, the binding domain includes or consists of the extracellular loop connecting the first and second transmembrane domains of ABCA1. Preferably, the binding domain includes or consists of amino acid residues from the human ABCA1 region from about position 602 to about position 620, or a functional equivalent or fragment thereof. In another preferred form of the invention the binding domain includes or consists of amino acid residues from the mouse ABCA1 region from position 542 to about position 560. In a highly preferred form of the invention the sequence is:

(SEQ ID NO:1)

CAIIRVLTGTEKKTGVYMQ

or a functional equivalent or fragment thereof.

In another form of the invention, the binding domain includes or consists of a part of the extracellular loop connecting the 7^{th} and 8^{th} transmembrane domains of ABCA1. In a preferred form of the invention the binding domain includes or consists of amino acid residues from the mouse ABCA1 region from about position 1251 to about position 1390 or a functional equivalent or fragment thereof. Preferably, the binding domain includes or consists of amino acid residues from the human ABCA1 region from about position 1311 to about position 1450 or a functional equivalent or fragment thereof. In a highly preferred form of the invention the sequences is:

(SEQ ID NO:2)

MDGKGSYQLKGWKLTQQQFVALLWKRLLIARRSRKGFFAQIVLPAVFVCI
ALVFSLIVPPFGKYPSLELQPWMYNEQYTFVSNDAPEDMGTQELLNALTK
DPGFGTRCME GNPIPDTPCLAGEEDWTISPVPQSIVDLFQ.

or a functional equivalent or fragment thereof.

The skilled person will understand that it is not strictly necessary that the binding domains described herein have protein sequences identical to those disclosed herein. There may be difference from species to species or even individual to individual in the amino acid sequences that may define the binding domains described herein.

Accordingly, the binding domains of the present invention include “functional equivalents” of the sequence such as mutants, including insertion, deletion or substitution of amino acids. Amino acid insertional derivatives include intrasequence insertions of single or multiple amino acids. Deletional variants are characterized by the removal of one or more amino acids from the sequence. Substitutional amino acid variants are those in which at least one residue in the sequence has been removed and a different residue inserted in its place. An example of substitutional amino acid variants are conservative amino acid substitutions. Accordingly the following amino acid substitutions are anticipated:

Lysine replaced with an amino acid selected from the group consisting of beta lysine, arginine, beta arginine, histidine and beta histidine.

Arginine replaced with an amino acid selected from the group consisting of beta arginine, lysine, beta lysine, histidine and beta histidine.

Histidine replaced with an amino acid selected from the group consisting of beta histidine, lysine, beta lysine, arginine and beta arginine.

Aspartic acid replaced with an amino acid selected from the group consisting of beta aspartic acid, glutamic acid and beta glutamic acid.

Glutamic acid replaced with an amino acid selected from the group consisting of beta glutamic acid, aspartic acid and beta aspartic acid.

Glycine replaced with an amino acid selected from the group consisting of asparagine, beta asparagine, glutamine, beta glutamine, cysteine, beta cysteine, serine, beta serine, threonine, beta threonine, tyrosine and beta tyrosine.

Asparagine replaced with an amino acid selected from the group consisting of glycine, beta asparagine, glutamine, beta glutamine, cysteine, beta cysteine, serine, beta serine, threonine, beta threonine, tyrosine and beta tyrosine.

Glutamine replaced with an amino acid selected from the group consisting of glycine, asparagine, beta asparagine, beta glutamine, cysteine, beta cysteine, serine, beta serine, threonine, beta threonine, tyrosine and beta tyrosine.

Cysteine replaced with an amino acid selected from the group consisting of glycine, asparagine, beta asparagine, glutamine, beta glutamine, beta cysteine, serine, beta serine, threonine, beta threonine, tyrosine and beta tyrosine.

Serine replaced with an amino acid selected from the group consisting of glycine, asparagine, beta asparagine, glutamine, beta glutamine, cysteine, beta cysteine, beta serine, threonine, beta threonine, tyrosine and beta tyrosine.

Threonine replaced with an amino acid selected from the group consisting of glycine, asparagine, beta asparagine, glutamine, beta glutamine, cysteine, beta cysteine, serine, beta serine, beta threonine, tyrosine and beta tyrosine.

Tyrosine replaced with an amino acid selected from the group consisting of glycine, asparagine, beta asparagine, glutamine, beta glutamine, cysteine, beta cysteine, serine, beta serine, threonine, beta threonine, and beta tyrosine.

Alanine replaced with an amino acid selected from the group consisting of beta alanine, valine, beta valine, leucine, beta leucine, isoleucine, beta isoleucine, proline, phenylalanine, beta phenylalanine, methionine, beta methionine, tryptophan and beta tryptophan.

Valine replaced with an amino acid selected from the group consisting of alanine, beta alanine, beta valine, leucine, beta leucine, isoleucine, beta isoleucine, proline, phenylalanine, beta phenylalanine, methionine, beta methionine, tryptophan and beta tryptophan.

Leucine replaced with an amino acid selected from the group consisting of alanine, beta alanine, valine, beta valine, beta leucine, isoleucine, beta isoleucine, proline, phenylalanine, beta phenylalanine, methionine, beta methionine, tryptophan and beta tryptophan.

Isoleucine replaced with an amino acid selected from the group consisting of alanine, beta alanine, valine, beta valine, leucine, beta leucine, beta isoleucine, proline, phenylalanine, beta phenylalanine, methionine, beta methionine, tryptophan and beta tryptophan.

Proline replaced with an amino acid selected from the group consisting of alanine, beta alanine, valine, beta valine, leucine, beta leucine, isoleucine, beta isoleucine, phenylalanine, beta phenylalanine, methionine, beta methionine, tryptophan and beta tryptophan.

Phenylalanine replaced with an amino acid selected from the group consisting of alanine, beta alanine, valine, beta valine, leucine, beta leucine, isoleucine, beta isoleucine, proline, beta phenylalanine, methionine, beta methionine, tryptophan and beta tryptophan.

Methionine replaced with an amino acid selected from the group consisting of alanine, beta alanine, valine, beta valine, leucine, beta leucine, isoleucine, beta isoleucine, proline, phenylalanine, beta phenylalanine, beta methionine, tryptophan and beta tryptophan.

Tryptophan replaced with an amino acid selected from the group consisting of alanine, beta alanine, valine, beta valine, leucine, beta leucine, isoleucine, beta isoleucine, proline, phenylalanine, beta phenylalanine, methionine, beta methionine, and beta tryptophan.

In another aspect the present invention provides a method of identifying a ABCA1 binding domain agonist or antagonist the method including exposing a potential agonist or antagonist to a ABCA1 binding domain or portion thereof, and determining the ability of the potential agonist or antagonist to bind to the ABCA1 binding domain or otherwise interfere with the binding of the ABCA1 binding domain with an associated molecule. In one form of the method the associated molecule is apoA-I.

Methods of determining the binding of an agonist or an antagonist may be conducted using biological assays, competition assays, binding assays, X-Ray crystallography and other methods known to the skilled artisan.

It will also be possible to identify of design agonists or antagonists of the ABCA1 binding domains described herein by X-Ray crystallography. X-ray crystallography relies on the observation that if a parallel X-ray beam is passed through a molecule, the X-rays will be deflected by electron dense regions. The scattering of the parallel X-ray beam will give a diagnostic deflection pattern, depending on the structure of the molecule. Unfortunately, molecules in solution are mobile and not aligned with their neighbouring molecules, meaning that the X-ray diffraction will be diffuse and non-interpretable. If, however, all of the molecules of a similar type are aligned in an orderly fashion for example, in a crystal, then X-ray diffraction will be orderly and the pattern of diffraction contains structural information about the molecule of interest. In X-ray crystallography, a crystal of, say, protein is bombarded with X-rays whilst it is rotated through an angle of 90°, thus allowing a ‘data set’ to be collected. Data is collected on an image plate or photographic film and interpreted by computer software because of the enormous number of data point and intensities collected. The three dimensional structure of the protein can be determined in this manner allowing for the rational design of compounds, agonists and antagonists which have the ability to modulate the binding of an agent to the binding domain.

In another aspect the present invention also provides a method for treating or preventing a disease related to a biological activity of ABCA1, the method including administering to a subject an effective amount of an ABCA1 agonist or antagonist as described herein.

Preferably, the disease is a disease related to cholesterol efflux. Such conditions result from an abnormally fast or abnormally slow rate of cholesterol efflux in a cell, as compared with that seen in the non-diseased condition. Preferably, the disease is selected from the group including but not limited to Tangier disease, coronary heart disease, atherosclerosis, and acquired immune deficiency syndrome.

With respect to the treatment of coronary heart disease, it has been previously shown that raising ABCA-1 production in macrophages locally reduces cholesterol deposition in coronary arteries without significantly raising plasma HDL cholesterol. In this instance, raising ABCA-1 expression is beneficial even in the absence of increased HDL cholesterol.

Reference herein to “treating” and “preventing” is to be considered in its broadest context. The term “treating” does not necessarily imply that a mammal is treated until total recovery. Similarly, “preventing” does not necessarily mean that the subject will not eventually contract a disease condition. Accordingly, treatment and prevention include amelioration of the symptoms of a particular condition or preventing or otherwise reducing the risk of developing a particular condition. The term “preventing” may be considered as reducing the severity of onset of a particular condition. “Treating’ may also reduce the severity of an existing condition or the frequency of acute attacks.

In terms of achieving an object of the present invention, an “effective amount” means an amount necessary to at least partly attain the desired response.

The term “subject” includes humans, primates, livestock animals (eg horses, cattle. sheep, pigs and donkeys), laboratory test animals (eg mice, rats, rabbits, guinea pigs), companion animals (eg dogs, cats) captive wild animals (eg kangaroos, deer, foxes), poultry birds (eg chickens, ducks, bantams, pheasants) reptiles and fish. Preferably, the subject is a human or a laboratory test animal. Even more preferably the subject is a human.

The method may include administering an agonist or antagonist of the ABCA1 binding domain as described above. The agonist or antagonist may be a peptide or an antibody, or a small inorganic molecule.

In a preferred form of the invention the ABCA1 agonist or antagonist is an antibody. Methods of making antibodies will be familiar to those skilled in the art and will be understood to further include the steps of inoculating an animal with a peptide molecule having the binding domain or a portion thereof as described above, fusing antibody-producing cells with a myeloma cell line and screening for a cell line that produces an antibody reactive with the binding domain or portion thereof, and harvesting antibodies from the cell line, testing for inhibition of high affinity binding and testing for inhibition or excitation of function. This may further include making small fragments of antibodies produced by the cell line capable of binding the binding domain or portion thereof. The cell line may conveniently be a mouse cell line and the method may include the further step of “humanising” the antibody fragments by replacing mouse sequences with human sequences in the non-binding regions. Humanising may be conducted by any methods known to the skilled addressee.

The antibody fragment may be a larger portion such as Fab fragments or much smaller fragments of the variable region. These fragments may be used as separate molecules or alternatively may form part of a recombinant molecule which is then used for therapeutic purposes. Thus for example the monoclonal antibody may be “humanised” by recombining nucleic acid encoding the variable region of the monoclonal antibody with nucleic acid encoding non-variable regions of human origin in an appropriate expression vector. Humanised monoclonal antibodies are contemplated to be useful in the therapeutic use of the antibodies disclosed herein.

According to one method for making human monoclonal antibodies, either whole human granulocytes or, preferably, membrane component fractions from such cells, are introduced into a severe-combined immunodeficient (SCID) mouse which has been repopulated with human lymphoid cells, in order to hyperimmunize the human lymphoid cells present in the mouse. See, e.g., Mosier et al, Nature, 335:256-9 (1988). The hyperimmunized cells are then isolated and cultured as immortalized cells, e.g., by Epstein-Barr virus (EBV) infection, and then fused to human myeloma cells to produce hybridomas. See, e.g., James et al., J. Immunol. Meth., 100:5-40 (1987). The resulting hybridomas are then screened, using, e.g., flow cytometry, to select hybridomas producing antibodies that bind to human granulocytes. Once a hybridoma is selected that produces useful quantities of an anti-granulocyte Monoclonal antibody, the culture supernatant is used as a source for purifying and recovering a pharmaceutically acceptable Monoclonal antibody by known methods. To the extent that a stable human Monoclonal antibody can be produced by such a technique or variant thereof, it will be an appropriate targeting component of a conjugate according to the invention.

The ABCA1 agonist and antagonist compounds of the present invention are not limited to antibodies reactive to the protein-binding domain or any portions thereof and which compete with the binding of substrates. Other compounds including small molecules or synthetic or natural chemical compounds capable of competing with the binding of a substrate to the binding domain or any portion thereof are also included in the present invention.

The present invention also provides compositions including the ABCA1 agonists and antagonists described herein, in combination with a carrier. Those skilled in the art will be able by routine experimentation to determine appropriate buffers, stabilisers, preservatives, excipients, adjuvants, solvents and the like suitable for preparing such a composition. The composition may further include other therapeutic compounds in addition to those defined above.

In another aspect the present invention provides a method of modulating a biological activity of ABCA1, the method including exposing ABCA1 to an agonist or antagonist described herein.

In another aspect the present invention provides a method for modulating cholesterol efflux or phospholipid efflux in a cell the method including exposing ABCA1 in the cell to an agonist or antagonist described herein.

In a further aspect the present invention provides a method of modulating the level of HDL in the blood of an animal the method including exposing the animal to an ABCA1 agonist or antagonist described herein.

Applicants have identified a number of antibodies capable of modulating a biological activity of ABCA1. Accordingly, a further aspect of the present invention provides an antibody selected from the group consisting of NDF4C2, NDF3F9, NDF2D12, or NDF6F1 or a functional equivalent or fragment thereof. Importantly, it has been found that these antibodies are able to bind to a binding domain of ABCA1, thereby modulating a biological activity.

Applicants demonstrate herein that the antibody NDF4C2 inhibited cholesterol efflux without affecting apoA-I binding. The antibody NDF3F9 inhibited apoA-I binding without affecting cholesterol efflux. These two antibodies were raised against the same fragment of ABCA1, but they belong to different isotypes and the way they bind to ABCA1 may differ. Consequently, they probably induce different conformational changes of the transporter and affect different functions of ABCA1. This finding indicates that one apoA-I molecule may be required to induce ABCA1-related changes in membrane structure enabling cholesterol to efflux to apoA-I.

The antibody NDF6F1 directed against a region of the fourth extracellular loop was shown to inhibit apoA-I binding, yet stimulated cholesterol efflux and reduced intracellular cholesterol content. The fourth extracellular loop as well as an epitope of NDF6F1 are located close to the PEST domain, a region responsible for ABCA1 internalization and degradation. As shown herein, preincubation of cells with NDF6F1 antibodies induced a similar (and non-additive) effect on cholesterol efflux as deletion of the PEST domain, indicating that a mechanism is similar to that of calpain-mediated degradation, may be involved. The effect of the antibody on ABCA1 stability was similar to the effect of apoA-I and apoA-I mimicking peptides suggesting that the mechanism may involve calpain-mediated degradation.

Without wishing to be limited by theory in any way, Applicant proposes that the antibody studies described herein support the following model of ABCA1/apoA-I mediated lipid efflux. According to this model three events can happen as a result of apoA-I binding to ABCA1. First, binding of apoA-I to ABCA1 may induce changes in the plasma membrane enabling cholesterol efflux. The antibody NDF4C2 blocks this process and reduces cholesterol efflux, without affecting binding of apoA-I. Second, binding of apoA-I to ABCA1 may lead to the internalization of apoA-I/ABCA1 complex, this event is blocked by the antibody NDF3F9. Internalization of apoA-I-ABCA1 complex may not be related to cholesterol efflux, but could be a different function, e.g. part of transcytosis of apoA-I. The third event is binding of apoA-I to the fourth extracellular loop of ABCA1 preventing its calpain-mediated proteolysis. The antibody NDF6F1 is likely binds to this site and acts as an agonist preventing ABCA1 degradation.

In addition to elucidating the biology of the ABCA1 molecule, provision of the antibodies described herein will form the basis for antibody therapeutics. The antibodies of the present invention may be used in vivo for therapeutic or diagnostic purposes. The antibody may be conveniently provided as an injectable preparation for mammalian use, preferably a sterile injectable preparation for human use, including: a sterile injectable solution containing an effective amount of the radiolabeled composite in a pharmaceutically acceptable sterile injection vehicle, preferably phosphate-buffered saline (PBS) at physiological pH and concentration. Other conventional pharmaceutically acceptable vehicles may be utilized as required for the site of parenteral administration.

A representative preparation to be parenterally administered in accordance with this invention will normally contain about 0.1 to 20 mg, preferably about 2 mg, of antibody, in a sterile solution which advantageously also contains, e.g., about 10 mg of human serum albumin (1% USP: Parke-Davis) per milliliter of 0.04M phosphate buffer (pH 7.4 Bioware) containing 0.9% sodium chloride.

Applicants describe development of four monoclonal antibodies against two regions of ABCA1. Two antibodies reacted with human, hamster and mouse ABCA1 reflecting an exceptional degree of conservation in ABCA1 sequence. The antibody NDF3F9 reacted with THP-1 cells probably reflecting higher levels of ABCA1 in these cells. The antibody NDF6F1 reacted with human and mouse, but not hamster ABCA1 indicating that hamster ABCA1 may have different sequence in the area representing epitope of this antibody. The antibodies did not neutralize the ability of ABCA1 to promote cholesterol efflux, moreover, they stimulated it.

Applicants demonstrated usefulness of the antibodies for quantitation of ABCA1 using Western blot or ELISA, for functional and morphological studies. The antibody can potentially be used for detection of certain ABCA1 mutations, especially those, which result in truncation of ABCA1.

Hybridoma cells used to produce the antibodies of the present invention have been deposited with The Korean Cell Line Research Foundation (Cancer Research Institute, Seoul National University College of Medicine, 28 Yongon-dong, Chongno-Gu, Seoul, 110-744, Korea). The International Depository Authority accepted the deposits on 8 Apr. 2004, and accession numbers were accorded as follows:

| | |
|---|---|
| NDF4C2 | KCLRF-BP-00094 |
| NDF3F9 | KCLRF-BP-00095 |
| NDF2D12 | KCLRF-BP-00097 |
| NDF6F1 | KCLRF-BP-00096 |

The skilled person is adequately enabled to produce and isolate an antibody from cultured hybridoma cells, including expansion of hybridoma clones from the deposited cells.

By way of explanation a number of methods are known for producing monoclonal antibodies. In one method, tissue culture adapted mouse myeloma cells are fused to antibody producing cells from immunized mice to obtain hybrid cells that produce large amounts of a single antibody molecule. In general, the antibody producing cells are prepared by immunizing an animal, for example, mouse, rat, rabbit, sheep, horse, or bovine, with an antigen. The immunization schedule and the concentration of the antigen in suspension is such as to provide useful quantities of suitably primed antibody producing cells. These antibody producing cells can be either spleen cells, thymocytes, lymph node cells and/or peripheral blood lymphocytes.

The antibody producing cells are then fused with myeloma cells, cell lines originating from various animals such as mice, rats, rabbits, and humans can be used, using a suitable fusion promoter. Many mouse myeloma cell lines are known and available generally from members of the academic community and various depositories, such as the American Type Culture Collection, (Manassas, Va.). The myeloma cell line used should preferably be medium sensitive so that unfused myeloma cells will not survive in a selective media, while hybrids will survive. The cell line most commonly used is an 8-azaguanine resistant cell line, which lacks the enzyme hypoxanthine-guanine-phosphoribosyl-transferase and therefore will not be supported by HAT (hypoxanthine-aminopterin-thymidine) medium. In general, the cell line is also preferably a “non-secretor” type, in that it does not produce any antibody. The preferred fusion promoter is polyethyleneglycol having an average molecular weight from about 1000 to about 4000. Other fusion promoters such as polyvinylalcohol, a virus or an electrical field can also be used.

The immortalized cells (hybridoma) must then be screened for those which secrete antibody of the correct specificity. The initial screening is generally carried out using an enzyme-linked immunosorbent assay (ELISA). Specifically, the hybridoma culture supernatants are added to microtitre plates which have been previously coated with the antigen, in this case purified ABCA1. A bound specific antibody from the culture supernatants can be detected using a labelled second antibody, for example, goat antimouse IgG labelled with peroxidase, which is commercially available. Cultures that are positive against ABCA1 antigen are then subjected to cloning by the limiting dilution method. Secondary hybridoma cultures are re-screened as described above. The cultures are then evaluated as to determine whether or not the antibody binds the antigen and to determine the kinetic profile of antigen binding. Selected cultures based on these results are subject to further cloning until culture stability and clonality are obtained. Immediately after hybridization, the fusion products will have approximately 80 chromosomes, and as these cells proceed to divide they will randomly lose some of these chromosomes. The cloning process is to select those cells which still have the chromosomes coding for antibody production. The cloning process is repeated until 100% of the sub-population exhibits the production of a specific antibody, which is indicative of the “stability” of the hybridoma. In addition, hybridoma culture wells often have multiple colonies some of which may be antibody non-producers. The cloning process allows the selection of a positive hybrid which is derived from a single cell.

The monoclonal antibody of the present invention can be produced either using a bioreactor or from ascites, both procedures of which are well known in the art.

The present invention is directed to the antibodies NDF4C2, NDF3F9, NDF2D12, and NDF6F1 as well as functional equivalents and fragments of the antibodies. As used herein the term “functional equivalent” is intended to include other molecules capable of binding ABCA1 derived directly or indirectly from any of the antibodies NDF4C2, NDF3F9, NDF2D12, and NDF6F1. Also included within the scope of this application are antibody fragments of the antibody such as Fab fragments, F(ab′)₂ fragments, Fv fragments and the like. These fragments can be obtained from the antibodies NDF4C2, NDF3F9, NDF2D12, and NDF6F1 by using techniques well known to those of skills in the art (Rousseaux et al. Methods Enzymology, 121:663-69, Academic Press, 1986).

It will also be understood that various mutations can be made to the amino acid sequence of an antibody while still resulting in an antibody that is capable of performing essentially the same or similar function to the antibodies NDF4C2, NDF3F9, NDF2D12, and NDF6F1. For example, an Fv fragment derived from an antibody of the present invention could be synthesised de novo using the amino acid sequence from a binding region of an antibody of the present invention, however with one or more alterations to the amino acid sequence. Alterations includes insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutantic acid; asparagine and glutamine; serine and threonine; lysine and arginine; and phenylalanine and tyrosine. Additions to amino acid sequences include fusions with other peptides, polypeptides or proteins.

A further embodiment of the present invention encompasses antibodies or fragments thereof capable of binding the same antigenic determinant as the antibody including, but not limited to, antibodies possessing the same antigenic specificity as the antibodies of the present invention but originating from a different species or having a different isotype or exhibiting different binding affinities. It is envisioned that class and isotype variants of the antibody of the present invention can be prepared using recombinant class switching and fusion techniques that are well known to those skilled in the art (see for example: Thammana et al. Eur. J. Immunol, 13:614, 1983; Oi et al., Biotechnologies, 4(3):214-221, Uu et al. Proc. Nat'l. Acad. Sci. (USA), 84:3439-43, 1987; Neuberger et al., Nature 312:604-608, 1984 and Spira et al. J. Immunol. Meth., 74:307-15, 1984).

Preferably, where antibody is NDF4C2 and NDF2D12 the antibody is IgM. In a further preferred form of the invention, wherein the antibody is NDF3F9 the antibody is IgG₃.

Also included in the scope of functional equivalents are chimeric or hybrid antibodies. The use of a chimeric “human” or “humanized” antibody in the present invention is motivated by the presence on the Fc portion of certain human immunoglobulin isotypes of regions that show high binding affinity to “Fc receptor” regions on certain populations or subpopulations of human mononuclear lymphoid cells. The generation of humanized antibodies has been discussed more fully infra.

The monoclonal antibodies of the present invention may be used in an immunoassay system for determining blood, serum, plasma or tissue levels of ABCA1. Current immunoassays utilize a double antibody method for detecting the presence of an analyte. These techniques are reviewed in “Basic Principals of Antigen-Antibody Reaction”, Elvin A. Labat, (Methods in Enzymology, 70, 3-70, 1980). Such systems are often referred to as fast format systems because they are adapted to rapid determinations of the presence of an analyte. The system requires high affinity between the antibody and the analyte. According to one embodiment of the present invention, the presence of ABCA1 is determined using a pair of antibodies, each specific for ABCA1. One of the pairs of antibodies is referred to herein as a “detector antibody” and the other of the pair of antibodies is referred to herein as a “capture antibody”. The monoclonal antibody of the present invention can be used as either a capture antibody or a detector antibody. The monoclonal antibody of the present invention can also be used as both capture and detector antibody, together in a single assay. One embodiment of the present invention thus uses the double antibody sandwich method for detecting ABCA1 in a sample of biological fluid. In this method, the analyte (ABCA1) is sandwiched between the detector antibody and the capture antibody, the capture antibody being irreversibly immobilized onto a solid support. The detector antibody would contain a detectable label, in order to identify the presence of the antibody-analyte sandwich and thus the presence of the analyte.

Common early forms of solid supports were plates, tubes or beads of polystyrene which are well known in the field of radioimmunoassay and enzyme immunoassay. More recently, a number of porous material such as nylon, nitrocellulose, cellulose acetate, glass fibres and other porous polymers have been employed as solid supports.

One embodiment of the present invention uses a flow-through type immunoassay device. Valkirs et al. (U.S. Pat. No. 4,632,901) discloses a device including antibody, specific to an antigen analyte, bound to a porous membrane or filter to which is added a liquid sample. As the liquid flows through the membrane, target analytes bind to the antibody. The addition of the sample is followed by the addition of a labelled antibody. The visual detection of the labelled antibody provides an indication of the presence of the target analyte in the sample.

Another example of a flow-through device is disclosed in Kromer et al. (EP-A 0 229 359), which described a reagent delivery system including a matrix saturated with a reagent or components thereof dispersed in a water soluble polymer for controlling the dissolution rate of the reagent for delivery to a reaction matrix positioned below the matrix.

In migration type assays, a membrane is impregnated with the reagents needed to perform the assay. An analyte detection zone is provided in which labelled analyte is bound and assay indicia is read. For example, see Tom et al. (U.S. Pat. No. 4,366,241), and Zuk (EP-A 0 143 574). Migration assay devices usually incorporate within them reagents which have been attached to coloured labels thereby permitting visible detection of the assay results without addition of further substances. See for example Bernstein (U.S. Pat. No. 4,770,853), May et al. (WO 88/08534), and Ching et al. (EP-A 0 299 428). The monoclonal antibody of the present invention can be used in all of these known types of flow-through devices.

Direct labels are one example of labels which can be used according to the present invention. A direct label has been defined as an entity, which in its natural state, is readily visible, either to the naked eye, or with the aid of an optical filter and/or applied stimulation, e.g. U.V. light to promote fluorescence. Among examples of coloured labels, which can be used according to the present invention, include metallic sol particles, for example, gold sol particles such as those described by Leuvering (U.S. Pat. No. 4,313,734); dye sole particles such as described by Gribnau et al. (U.S. Pat. No. 4,373,932) and May et al. (WO 88/08534); dyed latex such as described by May, supra, Snyder (EP-A 0 280 559 and 0 281 327); or dyes encapsulated in liposomes as described by Campbell et al. (U.S. Pat. No. 4,703,017). Other direct labels include a radionucleotide, a fluorescent moiety or a luminescent moiety. In addition to these direct labelling devices, indirect labels including enzymes can also be used according to the present invention. Various types of enzyme linked immunoassays are well known in the art, for example, alkaline phosphatase and horseradish peroxidase, lysozyme, glucose-6-phosphate dehydrogenase, lactate dehydrogenase, urease, these and others have been discussed in detail by Eva Engvall in Enzyme Immunoassay ELISA and EMIT in Methods in Enzymology, 70. 419-439, 1980 and in U.S. Pat. No. 4,857,453.

Other examples of biological diagnostic devices, which can be used for the detection of ABCA1, using the monoclonal antibody of the present invention, include the devices described by G. Grenner, P. B. Diagnostics Systems, Inc., in U.S. Pat. Nos. 4,906,439 and 4,918,025.

In one embodiment of the present invention, the diagnostic test uses a blood sample tube which is commonly used to draw blood samples from patients. The inside wall of the tube acts as a carrier for the monoclonal or polyclonal antibodies and required reagents or detection means, needed to produce a measurable signal. In this embodiment the capture antibody is immobilized onto the wall of the test tube. After the sample is drawn from the patient, the user simply shakes the sample with the detector antibody in the tube so that the detector antibody reacts with any ABCA1 in the blood. In this example the monoclonal antibody of the present invention can be either the capture antibody or the detector antibody. It may be necessary to use a sample wherein the red blood cells have been removed, so that the red blood cells will not interfere with the analysis of the results. If the analyte is present in the blood, it will be sandwiched between the capture antibody and the detector antibody which contains a suitable label for direct detection or reacts with the reagents in an indirect assay. The solid support (the test tube) can then be rinsed free of unbound labelled material. A variety of solid supports can be used according to this method, for example, test tube walls, plastic cups, beads, plastic balls and cylinders including microtitre plates, paper, and glass fibres.

There are currently available several types of automated assay apparatus which can undertake rapid format assays on a number of samples contemporaneously. These automated assay apparatus include continuous/random access assay apparatus. Examples of such systems include OPUS of PB Diagnostic System, Inc. and the IMX Analyzer by Abbott Laboratories of North Chicago, Ill. In general, a sample of the test fluid is typically provided in a sample cup and all the process steps including pipetting of the sample into the assay test element, incubation and reading of the signal obtained are carried out automatically. The automated assay systems generally include a series of work stations each of which performs one of the steps in the test procedure. The assay element may be transported from one work station to the next by various means such as a carousel or movable rack to enable the test steps to be accomplished sequentially. The assay elements may also include reservoirs for storing reagents, mixing fluids, diluting samples, etc. The assay elements also include an opening to permit administration of a predetermined amount of a sample fluid, and if necessary, any other required reagent to a porous member. The sample element may also include a window to allow a signal obtained as a result of the process steps, typically a fluorescent or a colorimetric change in the reagents present on the porous member to be read, such as by a means of a spectroscopy or fluorometer which are included within the assay system.

A further class of immunochemical analyzer systems, in which the monoclonal antibody of the present invention can be used, are the biosensors or optical immunosensor systems. In general an optical biosensor is a device which uses optical principles quantitatively to convert chemical or biochemical concentrations or activities of interest into electrical signals. These systems can be grouped into four major categories: reflection techniques; surface plasmon resonance; fibre optic techniques and integrated optic devices. Reflection techniques include ellipsometry, multiple integral reflection spectroscopy, and fluorescent capillary fill devices. Fibre-optic techniques include evanescent field fluorescence, optical fibre capillary tube, and fibre optic fluorescence sensors. Integrated optic devices include planer evanescent field fluorescence, input grading coupler immunosensor, Mach-Zehnder interferometer, Hartman interferometer and difference interfermoter sensors. These examples of optical immunosensors are described in general in a review article by G. A. Robins (Advances in Biosensors), Vol. 1, pp. 229-256, 1991. More specific description of these devices are found for example in U.S. Pat. Nos. 4,810,658; 4,978,503; 5,186,897; R. A. Brady et al. (Phil. Trans. R. Soc. Land. B 316, 143-160, 1987) and G. A. Robinson et al. (in Sensors and Actuators, Elsevier, 1992).

Another immunochemical analyzer is flow cytometry. In flow cytometry the sample containing the antigen is reacted with a fluorescently labelled form of the monoclonal antibody of the present invention. The sample is passed in front of a laser beam of a given wavelength capable of exciting the chromophore on the antibody. Each particle or cell having the antibody bound to it will fluoresce and will be detected. This technique allows the analysis of specific cell types and in particular of specific blood cell types. It is therefore useful for the detection of cells exhibiting the ABCA1 antigen.

In one embodiment of the present invention, ABCA1 is detected in a sample of blood, serum or plasma, using the monoclonal antibody of the present invention, in a device including a filter membrane or solid support with a detection section and a capture section. The detector section contains an antibody (a detector antibody), which will react with ABCA1. The detector antibody is reversibly immobilized onto the solid support and will migrate with the sample, when in use. It is preferred that the detector antibody is labelled, for example with a radionucleotide, an enzyme, a fluorescent moiety, luminescent moiety or a coloured label such as those described in the prior art, and discussed above. The capture section comprises a capture antibody, which is irreversibly immobilized onto the solid support. The antibodies, capture and detector antibody, and the necessary reagents are immobilized onto the solid support using standard art recognized techniques, as disclosed in the flow-through type immunoassay devices discussed previously. In general, the antibodies are absorbed onto the solid supports as a result of hydrophobic interactions between non-polar protein substructures and non-polar support matrix material.

According to this embodiment of the present invention, if ABCA1 is present, it will react with the detector antibody in the detector section and will migrate on the filter membrane towards the capture section where the analyte will further bind with the capture antibody. Thus, ABCA1 will be sandwiched between the capture antibody and the detector antibody, which contains a suitable label.

In this example of the present invention, if the detector antibody is labelled with a coloured label or an enzyme which will produce a coloured label, the patient's blood would first require centrifugation or some pre-filtering in order to remove the red blood cells so that the colour of the red blood cells will not interfere with the coloured labels. If radioactive labels or fluorescent labels are to be used, a pre-filtration or centrifugation step may not be required. In this embodiment, the monoclonal antibody of the present invention can be either the capture antibody or the detector antibody. In one embodiment, the monoclonal antibody of the present invention is a capture antibody. The detector antibody can be other ABCA1 monoclonal antibodies, or polyclonal anti-ABCA1 antibodies. Either chicken, rabbit, goat or mouse polyclonal antibodies can be used. Many such antibodies are known and can be prepared and labelled by known methods.

In a further embodiment of this invention the monoclonal antibody NDF4C2, NDF3F9, NDF2D12, and NDF6F1 can also be used to monitor patients that have or are at risk of developing a vascular disorder or a disorder related to an HDL receptor.

As would also be recognized by one of skill in the art, a base line level of ABCA1 may be present in normal patients. Thus, in the present invention, in certain embodiments, the levels of ABCA1 above or below normal will be determined. This can be accomplished by either comparing the results to the results of a normal patient, or adjusting the sensitivity of the immunoassay so that only values above a certain threshold will show as a positive result.

As will be clearly indicated in the examples infra, ABCA1 can be detected by Western Blotting. Thus, the monoclonal antibodies of the antibodies of the present invention, along with the embodiments described supra is particularly useful as a new diagnostic tool. The present invention also provides a composition including an antibody as described herein and an assay acceptable carrier. The skilled person will appreciate that the antibodies of the present invention may be used in the context of an assay selected from an assay such as Western Blot, immunofluorescence, flow cytometry, surface plasmon resonance, ELISA, and the like. In this context, the term “assay acceptable carrier” is intended to mean any solvent or solute that may be used to dissolve the antibody or maintain the antibody protein in a desired configuration. Solvents include water or alcohol. Solutes includes salts and other agents to maintain a desired ionic strength or pH. The term “assay acceptable carrier” is also intended to include blocking agents such as casein, bovine serum albumin and the like.

In another aspect the present invention also provides a method of isolating a subpopulation of cells from a population of cells, the method including exposing the population of cells to an antibody described herein, and separating the cells that specifically bind to the antibody from those that do not specifically bind to the antibody. The present invention also provides a subpopulation of cells isolated using the method. The subpopulation may predominantly include cells that do not specifically bind to an antibody as described herein. The subpopulation may predominantly include cells that specifically bind to an antibody as described herein.

In a further aspect the present invention also provides a method for isolating an ABCA1 molecule including the use of an antibody described herein. Affinity chromatography where the antibody is coupled to a stationary chromatography support is one method that could be used to achieve isolation.

In a further aspect the present invention provides an anti-idiotype antibody that is substantially a “mirror-image” of any of the antibodies NDF4C2, NDF3F9, NDF2D12, and NDF6F1. The variable region of an anti-idiotype antibody provides an approximate three-dimensional representation of the region on the target molecule to which the antibody NDF4C2, NDF3F9, NDF2D12, and NDF6F1 binds.

The present invention will now be more fully described by reference to the following non-limiting examples.

### EXAMPLES

### Example 1

### Materials and Methods

Antigens

Eighteen amino acid peptide corresponding to the sequence of human ABCA1 602-620 was synthesized commercially (Mimotopes, Clayton, Australia). Cystein was attached to the amino-terminal end to enable coupling and absorption on ELISA plates. The final sequence was: CAIIRVLTGTEKKTGVYMQ. Purity of the peptide was 60-80%. Peptide-KLH and peptide-BSA conjugates were synthesized via the EDC and S—NHS reaction using Imject Maleimide Activated mcKLH kit (Pierce, Rockford, II) according to manufacturer instructions.

A fragment of mouse ABCA1 corresponding to amino acids 1251-1390 was expressed as fusion protein with MBP using E coli expression system. The sequence used for expression was: MDGKGSYQLK GWKLTQQQFV ALLWKRLLIA RRSRKGFFAQ IVLPAVFVCI ALVFSLIVPP FGKYPSLELQ PWMYNEQYTF VSNDAPEDMG TQELLNALTK DPGFGTRCME GNPIPDTPCL AGEEDWTISP VPQSIVDLFQ.

Immunization and Fusion

Antibody production against hABCA1 peptide-KLH and mABCA1 fragment-MBP was initiated by emulsifying antigen in Freund's complete adjuvant and administering them intraperitoneally to 6-8-week-old female BALB/c mice. The amount of antigen administered was 20, 50 and 100 μg per animal. Two booster injections were performed in two-week intervals using Freund's incomplete adjuvant. Blood samples were taken from the saphenous vein. The bleeds were tested using ELISA. The best responding mice were intravenously injected once more with antigens without adjuvant. Four days later splenectomy was done and splenocytes were isolated using Lympholyte M (Cedarlane, Ontario, Canada), collected and fused with Sp2/0-Ag14 mouse myeloma cells. Fusion and hybridoma selection were done as described by Kohler and Milstein (Kohler, G., and Milstein, C. (1975) Nature 256, 495-497).

Cell Culture

Sp2/0-Ag14 myeloma and hybridoma cells were cultured in RPMI Medium 1640 supplemented with 10% fetal bovine serum, 50 U/ml penicillin, 50 μg/ml streptomycin, 2 mM glutamine. Hybridoma cell culture supernantants were tested in ELISA on plates coated with ABCA1 peptide-BSA or ABCA1 fragment-MBP or MBP alone). Responding cells were cloned at least twice by limiting dilution. The subclasses of all the monoclonal antibodies were determined using isotyping kit (Sigma, St Louis). Cells were propagated in a commercial facility (Chemicon Australia) or in CELLine CL350 bioreactor (Integra Bioscience, Switzerland) and antibodies were purified by affinity chromatography with protein G (for IgG) or using HiTrap IgM Purufication HP column (Amersham Biosciences) (for IgM).

HEK293 cells were transiently transfected with mouse ABCA1 using Lipofectamine as described by the manufacturer (Invitrogen). Prior to experiments THP-1 cells were differentiated by treatment with PMA and expression of ABCA1 was stimulated by treatment with LXR agonist T-0901317 for 18 h.

Mouse macrophages RAW 264.7 were cultured as described previously (Escher, G., Hoang, A., Georges, S., Tchoua, U., El-Osta, A., Krozowski, Z., and Sviridov, D. (2005) J. Lipid Res. 46, 356-365).

Human macrophages THP-1 were cultured as described previously (Sviridov, D., Miyazaki, O., Theodore, K., Hoang, A., Fukamachi, I., and Nestel, P. (2002) Arterioscler Thromb Vasc Biol 22, 1482-1488). Prior to experiments, THP-1 cells were differentiated by treatment with 100 μg/mL PMA (Sigma) for 72 h and when indicated expression of ABCA1 was stimulated by treatment with the LXR agonist TO-901317 (final concentration 4 μmol/L) for 18 h. HeLa cells were transfected using Effectene (Qiagen) according to manufacturer's instructions. Plasmid with wild type ABCA1 was obtained from Dr. G. Chimini and ΔPEST-ABCA1 plasmid was a kind gift of Dr. A. Tall. Mock transfection was done with the pCMV-β-gal plasmid.

Lipoproteins

Apolipoprotein A-I was isolated from human plasma as described previously (Sviridov, D., Pyle, L., and Fidge, N. (1996) J. Biol. Chem. 271, 33277-33283). LDL was purified from human plasma by sequential centrifugation and acetylated as described by Basu et al. (Basu, S. K., Goldstein, J. L., Anderson, G. W., and Brown, M. S. (1976) Proc Natl Acad Sci USA 73, 3178-3182).

Immunoblotting

Cells grown in 75 cm² flasks (approximately 15×10⁶ cells per flask) were treated as indicated, washed and harvested. Soluble membrane protein fractions were isolated as described by Yamauchi et al. (Yamauchi, Y., Abe-Dohmae, S., and Yokoyama, S. (2002) Biochim Biophys Acta 1585, 1-10). Total protein was determined by the Bradford assay (Pierce). Proteins were separated on a 6% SDS-PAGE followed by immunoblotting. In brief, PVDF membranes were blocked with 2.5% skim milk solution, washed, incubated 1 h at room temperature with non-diluted hybridoma cell culture supernantants, washed, incubated with biotinylated goat anti-mouse IgG or IgM (Chemicon, CA), washed and incubated with streptavidin-HRP conjugate (Chemicon, CA). Bands were visualized by SuperSignal West Pico Chemiluminescent Substrate kit (Pierce, Rockford, Ill.) and the relative intensities of the bands were quantitated by densitometry.

Cholesterol and Phospholipid Efflux

Cholesterol and phospholipid efflux experiments were conducted as described previously (Sviridov, D., Miyazaki, O., Theodore, K., Hoang, A., Fukamachi, I., and Nestel, P. (2002) Arterioscler Thromb Vasc Biol 22, 1482-1488). Briefly, THP-1 or HeLa cells were grown in 12-well plates and when indicated ABCA1 expression in THP-1 cells was boosted by adding 4 μmol/L of LXR agonist TO-901317. Cellular cholesterol or phospholipids were labeled by incubation in serum-containing medium with [1α,2α(n)-3H]-cholesterol (Amersham, specific radioactivity 1.81 TBq/mmol, final radioactivity 0.5 MBq/ml) or [methyl-¹⁴C] choline (0.2 MBq/ml) for 48 h in a CO₂ incubator. Cells were then washed and incubated for 18 h at 37° C. with serum-free medium containing indicated concentrations of the antibodies. Cells were washed and incubated for 3 h at 37⁰ C with serum-free medium containing 30 μg/ml of lipid-free apoA-I. For cholesterol efflux analysis, the medium was collected, centrifuged for 15 min at 4° C. at 10,000×g and aliquots of supernatant were counted in a β-counter. Cells were harvested and radioactivity was counted. For phospholipid efflux, phospholipids were isolated from medium and cells by TLC as described previously (Sviridov, D., Hoang, A., Huang, W., and Sasaki, J. (2002) J. Lipid. Res. 43, 1283-1292) and radioactivity was counted.

Cholesterol and phospholipid efflux was expressed as a proportion of [³H]cholesterol or [¹⁴C] phospholipid transferred from cells to medium.

Confocal Microscopy

THP-1 cells were grown on sterile glass cover slips to approximately 60% confluence and differentiated by incubation for 48 h with 100 ng/ml of PMA. ABCA1 expression was activated by incubation for 18 h with 4 μmol/L of TO-901317. Cells were then fixed for 10 min with 4% paraformaldehyde, permeabilized with 0.2% Triton X-100, washed with PBS, blocked for 30 min with PBS containing 10% goat serum, washed again and stained. The primary antibodies were either monoclonal anti-ABCA1, polyclonal anti-ABCA1 (Novus Biologicals), or either monoclonal or polyclonal anti-calnexin antibodies. Secondary antibodies were either Alexa Fluor 488 labeled goat anti-mouse IgM or IgG antibody, or Texas Red labeled goat anti-rabbit antibody (Molecular Probes). Cover slips were then mounted onto glass slides and viewed using a Zeiss META confocal microscope.

Binding Assay

ApoA-I was iodinated using Iodobeads (Pierce) according to the manufacturer's instructions. THP-1 cells were grown as described above in 12-well plates and when indicated the expression of ABCA1 was boosted by 4 μmol/L of LXR agonist TO-901317. Cells were incubated for 2 h at 37° C. in serum-free medium containing 0.1% BSA (essentially fatty-acid free, Sigma), 5 μg/ml of ¹²⁵I-apoA-I in the presence or absence of 250 μg/ml of unlabeled apoA-I. Medium was used to assess ¹²⁵I-apoA-I degradation. Cells were washed 3 times with serum-free medium containing 0.1% BSA and once with Ca⁺⁺, Mg⁺⁺ free PBS and treated with 0.05% trypsin/0.002% EDTA for 5 min at 37° C. The reaction was stopped by adding 100 μl of fetal calf serum. Radioactivity in the medium (binding) and cells (internalization) was determined with γ-counter. Degradation was determined as non-iodine, trichloroacetic acid-soluble radioactivity in the incubation medium as previously described (Sviridov, D. D., Safonova, I. G., Tsybulsky, V. P., Talalaev, A. G., Preobrazensky, S. N., Repin, V. S., and Smirnov, V. N. (1987) Biochim Biophys Acta 919, 266-274).

Analysis of Cell Surface ABCA 1

For cell surface ABCA1 analysis, cells were first biotinylated with 10 mmol/L of Sulfo-NHS-SS-Biotin (Pierce Chemical Co., Rockford, Ill., USA) at 4° C. for 30 minutes and then lysed with RIPA buffer at 4° C. After centrifugation, the supernatant of cell lysate was incubated with UltraLink Immobilized Streptavidin beads (Pierce) overnight at 4° C. Following centrifugation and washing, the collected beads were resuspended in SDS-PAGE sample buffer with 100 mmol/L 2-mercaptoethanol and boiled for 2 min. ABCA1 was detected by Western blot using NDF4C2 antibody.

In-Cell Western Blot

THP-1 cells were differentiated and ABCA1 expression was boosted by incubation with TO-901317 (4 μmol/L) for 18 h. Cells were then washed and incubated for the indicated periods of time with serum-free medium (control), fresh TO-901317 (4 μmol/L), apoA-I (50 μg/ml) or NDF6F1 or NDF4C2 antibodies (10 μg/ml). Cells were washed, fixed with 3.7% formaldehyde in PBS for 20 min and permeabilized with three 10-min washes with 0.1% Triton X-100 in PBS. LI-Cor blocking buffer was added for 2 h followed by overnight incubation with monoclonal NDF4C2 anti-ABCA1 antibodies and rabbit polyclonal anti-β-actin antibodies in 50% blocking buffer in PBS supplemented with 0.2% Tween20. After three washes, secondary detection was carried out using two species-specific infrared fluorescent dye conjugated antibodies in 1:1 LBB buffer and PBS supplemented with 0.4% Tween20. After a 1 h incubation, targets were simultaneously visualized and quantitated using the Odyssey Infrared Imaging Scanner (Li-Cor, Lincoln, NB) with 680-nm fluorofore emitting a red color and the 800-nm fluorofore emitting a green color. ABCA1 abundance was presented relative to abundance of β-actin.

Cholesterol Biosynthesis and Esterification

To assess cholesterol biosynthesis and esterification, macrophages were incubated for 2 h at 37° C. with [³H]acetic acid sodium salt (Amersham, specific activity 370 GBq/mmol; final radioactivity 740 MBq/ml) and [¹⁴C]oleic acid (Amersham, specific activity 2.22 GBq/mmol; final radioactivity 0.185 MBq/ml) complexed to BSA (Sigma, essentially fatty acid free). Cells were washed and lipids were extracted and analyzed by TLC as described previously (Sviridov, D., and Fidge, N. (1995) J. Lipid Res. 36, 1887-1896). Spots of cholesterol and cholesteryl oleate were identified by standards (Sigma), scraped and counted in a β-counter.

Statistical Analysis

All experiments were reproduced 2-4 times and representative experiments are shown. Unless otherwise indicated, experimental groups consisted of quadruplicates; means±SEM are presented. The Student's t-test was used to determine statistical significance of the differences.

### Example 2

### Selection of Putative ABCA1 Binding Domains

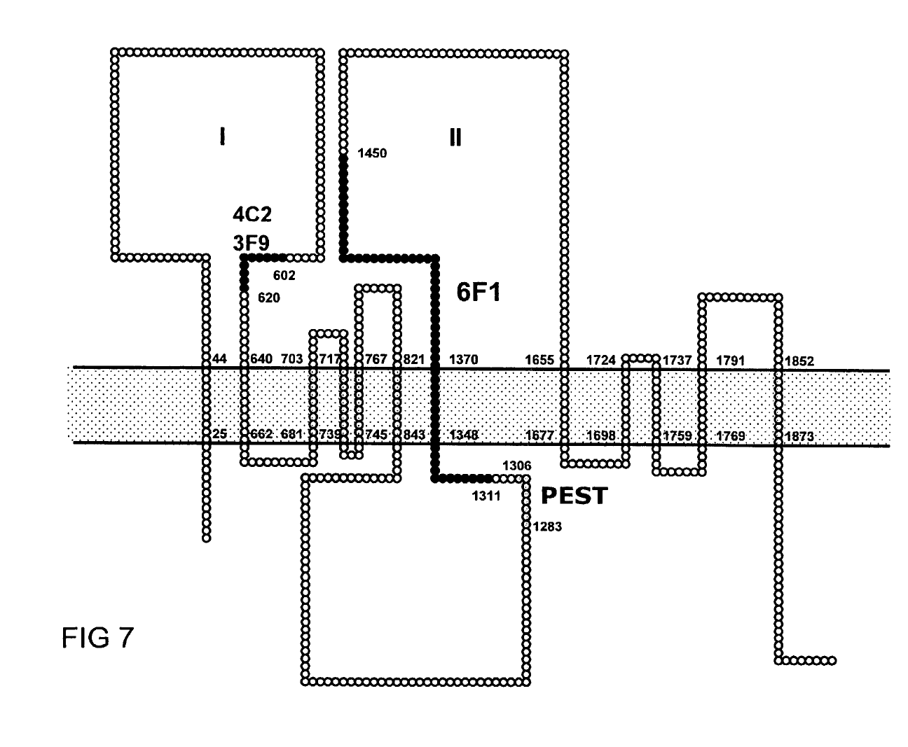

Two fragments of ABCA1 were used as antigens for production of monoclonal antibodies. First is 18 amino acid peptide corresponding to residues 602-620 of human ABCA1 and to residues 542-560 of mouse ABCA1. The difference between human and mouse sequences in the selected region is only one amino acid (S for T at position 10). The region represents a part of extracellular loop connecting first and second transmembrane domains of ABCA1. The second fragment is 140 amino acid peptide corresponding to residues 1311-1450 of human ABCA1 and 1251-1390 of mouse ABCA1. FIG. 7 shows a proposed topography of ABCA1 and location of two ABCA1 fragments used for development of the antibodies. The difference between human and mouse sequences is 9 amino acids; mouse sequence was used. The region represents a part of extracellular loop connecting 7th and 8th transmembrane domains of ABCA1. The following criteria were used to select the regions: i) both regions were extracellular; ii) when sequence of ABCA1 was analyzed using Jameson-Wolf antigenic index, the regions were the most antigenic regions of ABCA1 and iii) they did not contain consensus glycosylation sites.

### Example 3

### Generation of Antibodies

Mice injected with peptide-hABCA1 coupled to KLH gave modest immune response, but after fusion multiple clones positive to peptide-hABCA1 coupled to BSA were produced. Three clones with strongest response were selected and further subcloned. The monoclonal antibody produced by these clones were named NDF4C2, NDF3F9. NDF4C2 and NDF2D12 were determined to be IgM and NDF3F9 was IgG₃.

Mice injected with fragment of mABCA1 fused with MPB gave strong immune response, however after fusion a single clone was positive for ABCA1-MBP and negative for MBP alone. This clone was sub-cloned and the antibody named NDF6F1.

### Example 4

### Antibody Testing

The antibodies were tested using three sources of ABCA1: HEK293 cells transiently transfected with mouse ABCA1, CHOP cells, which express large quantities of hamster ABCA1 and activated THP-1 cells, human macrophages expressing human ABCA1. The advantage of this approach is that macrophages naturally express ABCA1 and play a key role in atherosclerosis.

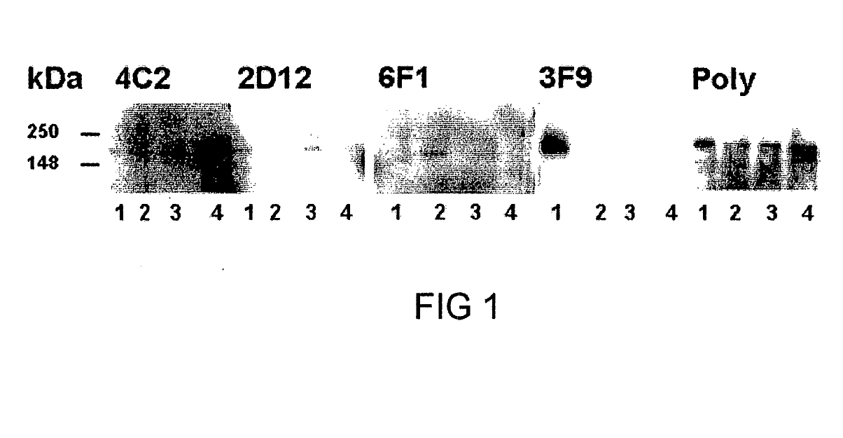

Antibodies NDF4C2 and NDF2D12 stained two bands in all cell types (FIG. 1). One band has the molecular weight of ABCA1 and was migrating at the same position as ABCA1 stained with polyclonal anti ABCA1 antibody. The second band had slightly lower molecular and may represent a result of post-translational processing of ABCA1. Antibody NDF3F9 gave one strong band corresponding to ABCA1 in THP-1 cells only (FIG. 1). Antibody NDF6F1 revealed two bands in all cells except CHOP cells. (FIG. 1).

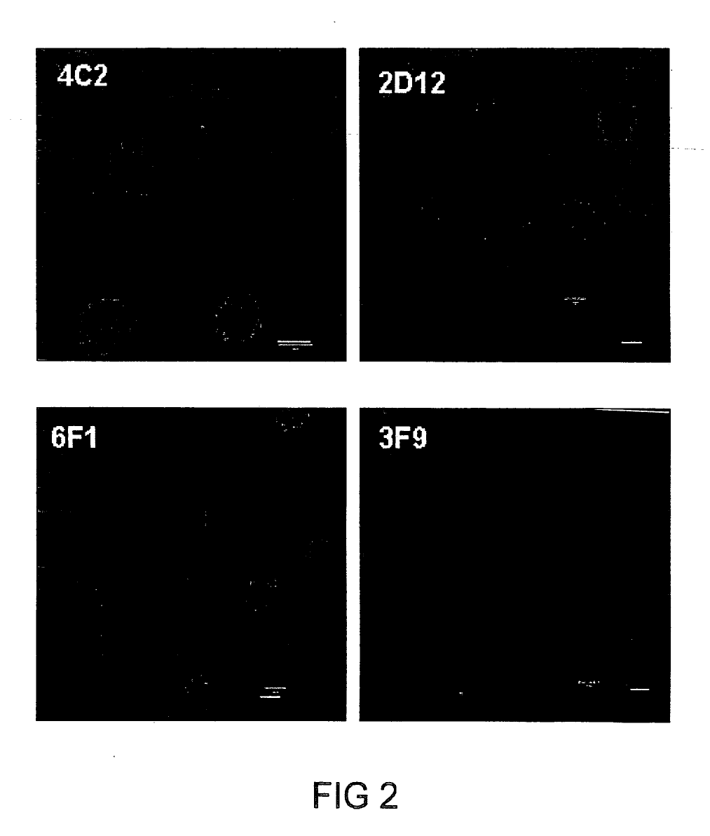

THP-1 cells were treated with the antibodies and distribution of ABCA1 was studied using confocal microscope. All four antibodies stained both endoplasmic reticulum and cell plasma membrane (FIG. 2), a localization of ABCA1 similar to that demonstrated by others.

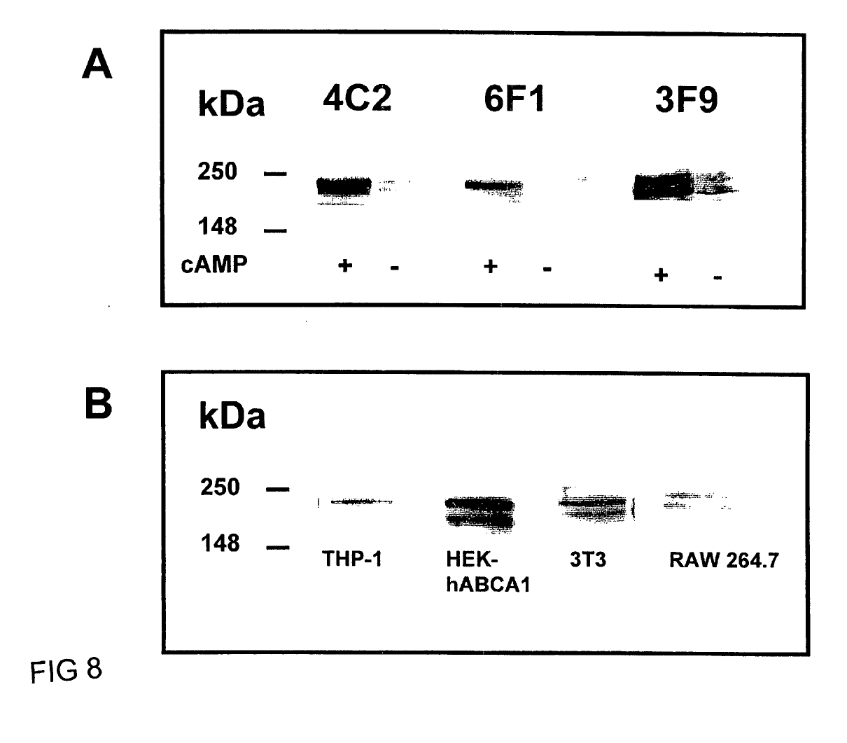

The antibodies were also tested using ABCA1 from RAW 264.7 mouse macrophage cell line activated or not with 0.3 mmol/L cAMP. cAMP is known to induce ABCA1 expression and to stimulate ABCA1-dependent cholesterol efflux in mouse macrophages (Zheng, P., Horwitz, A., Waelde, C. A., and Smith, J. D. (2001) Biochim Biophys Acta 1534, 121-128.). In an immunoblot analysis all three antibodies gave strong bands corresponding to ABCA1 (FIG. 8A). The amount of ABCA1 in cells stimulated by cAMP was 5-10 times higher compared to non-activated cells (FIG. 8A). The antibodies were then tested with ABCA1 from various cell types, namely human monocyte-macrophages THP-1, HEK293 cells transiently transfected with human ABCA1, and mouse fibroblasts 3T3. In all cell types the band similar to that in RAW 264.7 cells was detected by the NDF4C2 antibody (FIG. 8B). Two other antibodies also detected similar bands in these cell types (not shown).

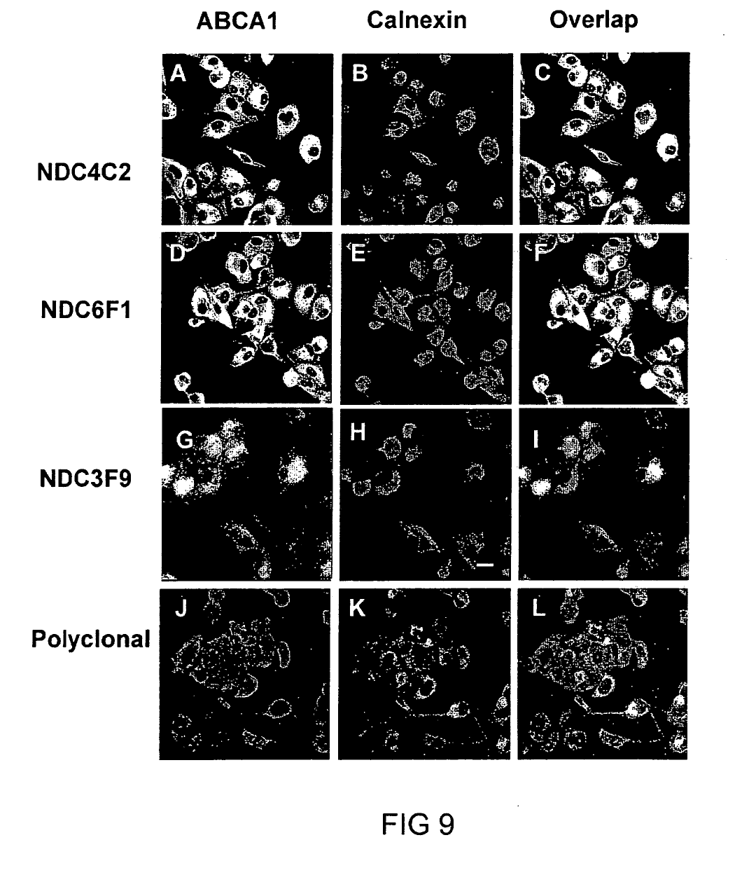

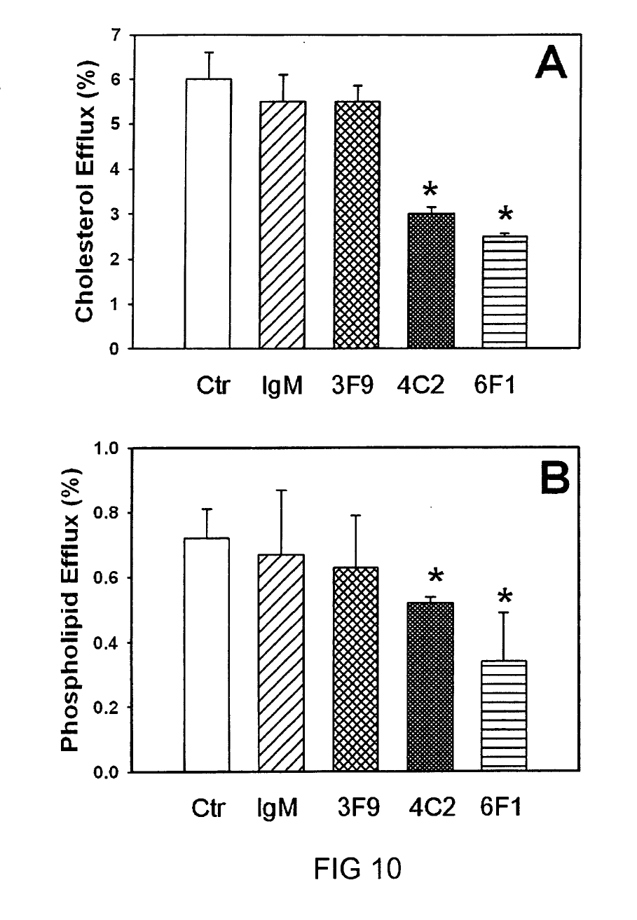

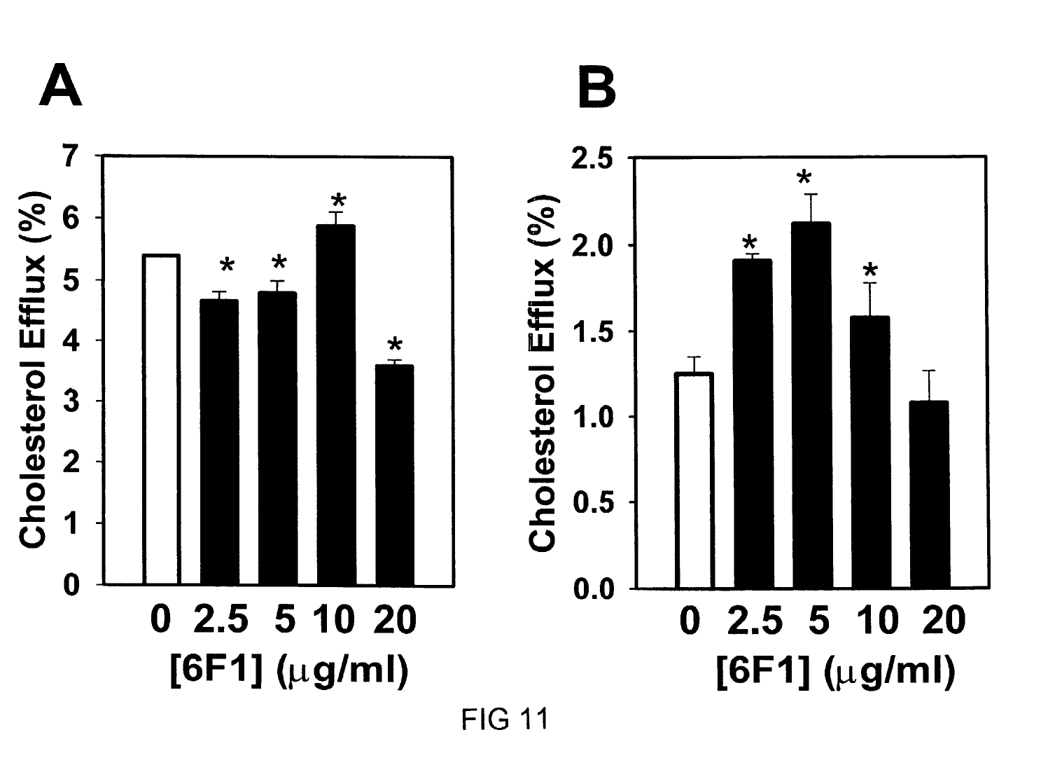

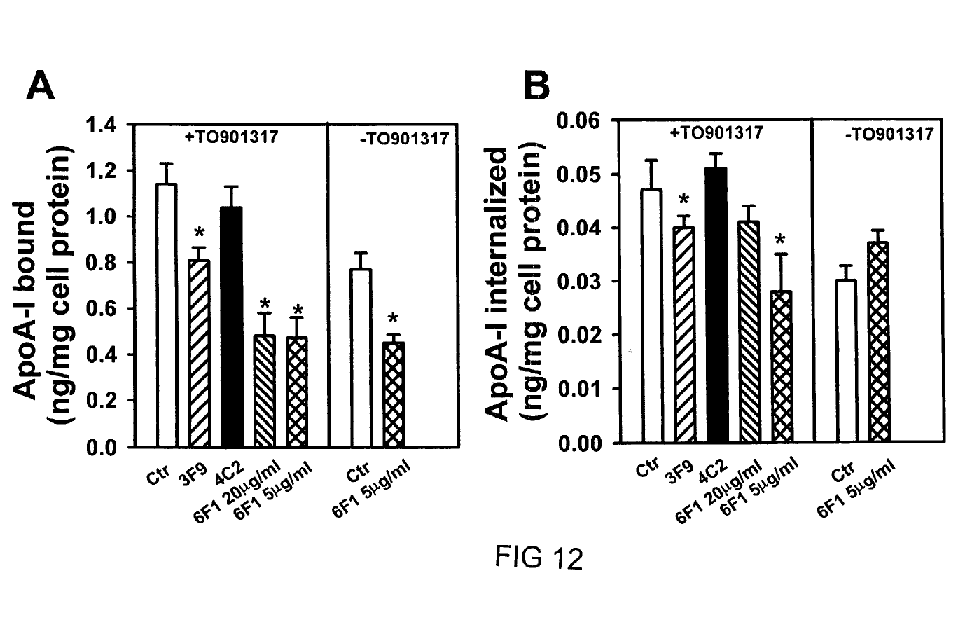

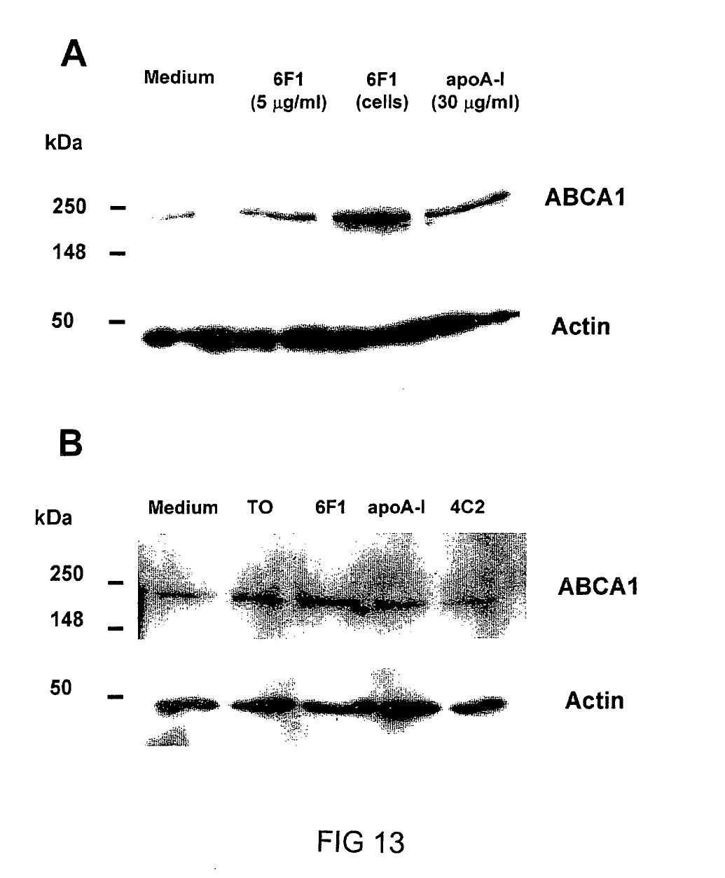

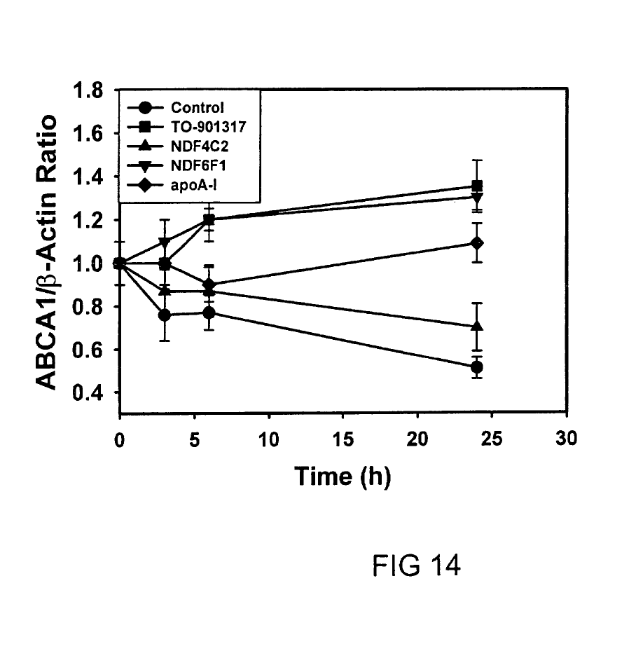

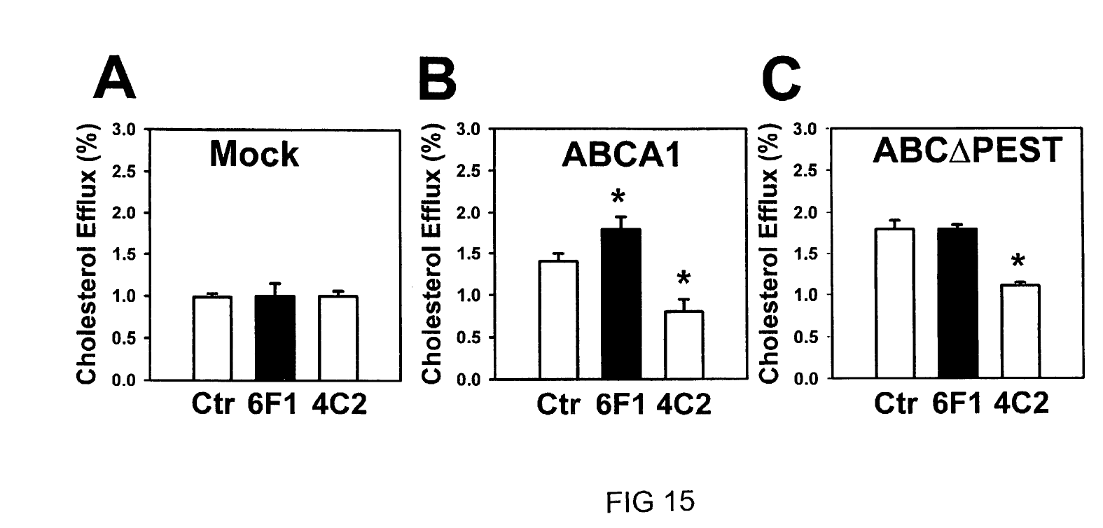

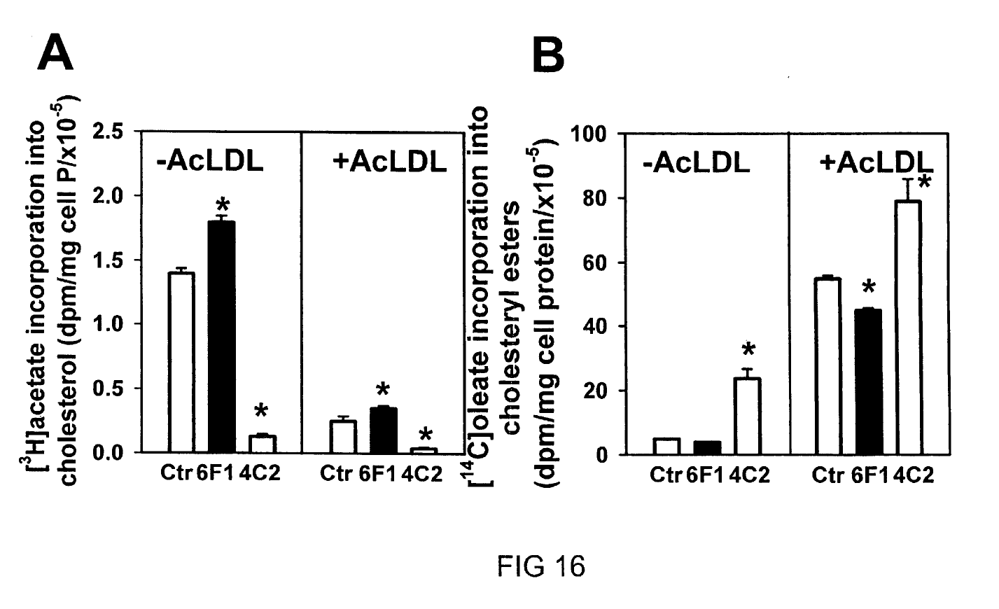

To test if the antibodies can be used in immunofluorescence microscopy, differentiated THP-1 cells treated with TO-901317 were stained with antibodies and the distribution of ABCA1 was studied using confocal microscopy. To assess the localization of ABCA1, cells were also stained with the antibodies against calnexin, a marker of endoplasmic reticulum. All three antibodies stained ABCA1 in both intracellular compartments and plasma membrane (FIGS. 9A, 9D, 9J). There was a partial overlap between the intracellular portion of ABCA1 and calnexin (FIG. 9A-91). The staining was similar to that observed with widely used polyclonal anti-ABCA1 antibodies (FIG. 9J-9L, note that colors are reversed as polyclonal antibodies were used to detect ABCA1 and monoclonal antibodies were used to detect calnexin). There was only a very weak staining when cells were not activated by TO-901317 and no staining when first antibodies were omitted (not shown).

### Example 5

### Effect of Antibodies on Cholesterol and Phospholipid Efflux

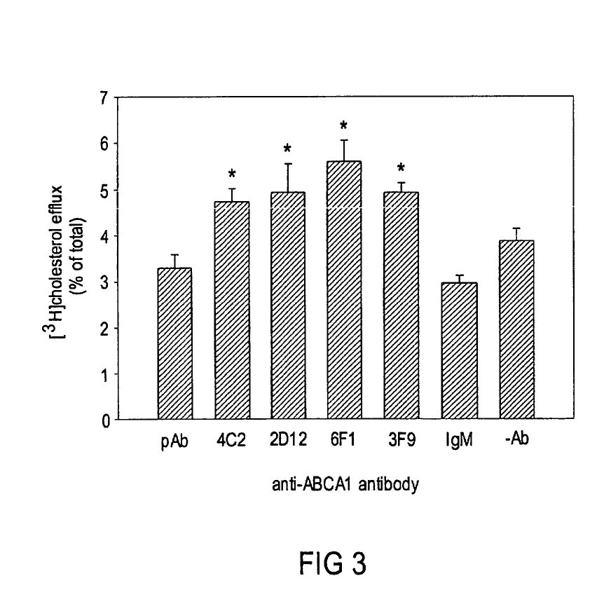

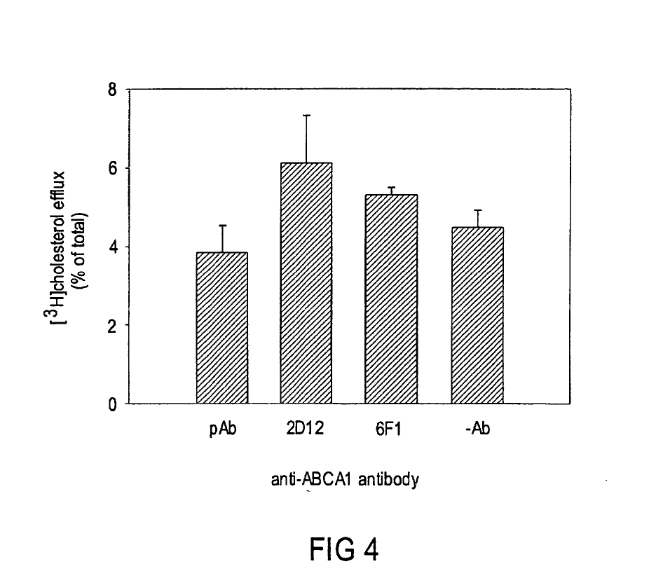

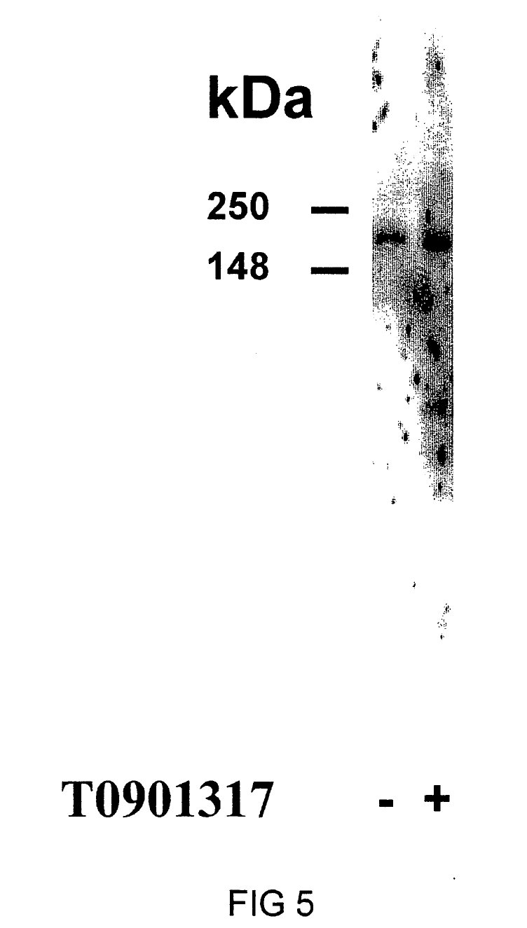

To test if antibodies can affect ABCA1 function, THP-1 cells were labeled with [3H]cholesterol and pre-incubated for 4 h with serum-free medium contained each of the antibody. Cholesterol efflux to lipid-free apoA-I was then tested as described in Example 1. All monoclonal antibodies stimulated cholesterol efflux while polyclonal antibody had no effect. (FIG. 3).

To further test if the antibodies can affect ABCA1-dependent cholesterol efflux, ABCA1 expression in THP-1 human macrophages was induced by incubation with LXR agonist TO-901317. Cells were labeled with [³H]cholesterol, pre-incubated for 18 h in serum-free medium containing 20 μg/ml of each of the antibodies and cholesterol efflux to lipid-free apoA-I was tested as described in “Materials and Methods”. Neither non-specific mouse IgM nor the antibody NDF3F9 affected cholesterol efflux, while the antibodies NDF4C2 and NDF6F1 inhibited cholesterol efflux by approximately 50% (p<0.0001) (FIG. 10A). The effect of the antibodies on phospholipid efflux was similar to their effect on cholesterol efflux (FIG. 10B). When the effect of the antibodies on cholesterol efflux from THP-1 cells not stimulated with TO-901317 was studied, again NDF3F9 was not active while NDF4C2 inhibited cholesterol efflux (not shown). Surprisingly, however, the antibody NDF6F1 stimulated cholesterol efflux. In a separate experiment a dose dependence of the effect of the antibody NDF6F1 on cholesterol efflux from THP-1 cells, treated or not treated with TO-901317, was studied. In both cases a bell-shaped response was observed (FIG. 11). When THP-1 cells were activated, low concentrations of NDF6F1 either slightly inhibited or slightly stimulated cholesterol efflux, while high concentration clearly inhibited cholesterol efflux (FIG. 11A). When ABCA1 in THP-1 cells was not induced, low concentrations of NDF6F1 clearly stimulated cholesterol efflux while higher concentrations did not affect it (FIG. 11B). The bell-shaped response suggests that the antibody may have two opposing effects on cholesterol efflux with the overall effect depending on a ratio between the concentration of the antibody and abundance of ABCA1. Similar effects were demonstrated when the antibodies were tested using RAW 264.7 mouse macrophages instead of THP-1 human macrophages (not shown)

### Example 6

### Effect of Pre-Incubated Antibodies on Cholesterol Efflux

This Example investigated the effect of pre-incubation with antibodies overnight followed by measurement of cholesterol efflux in their absence. THP-1 cells were labeled with [³H]cholesterol and incubated with 100 mcg/ml of each antibody for 18 h at 37° C. The antibodies were washed out and cells were incubated with 50 mcg/ml of lipid-free apoA-I for 2 h at 37° C. The amount of labeled cholesterol moved from cells to medium was measured as described in Example 1.

### Example 7

### Localization of ABCA1 in RAW Macrophages

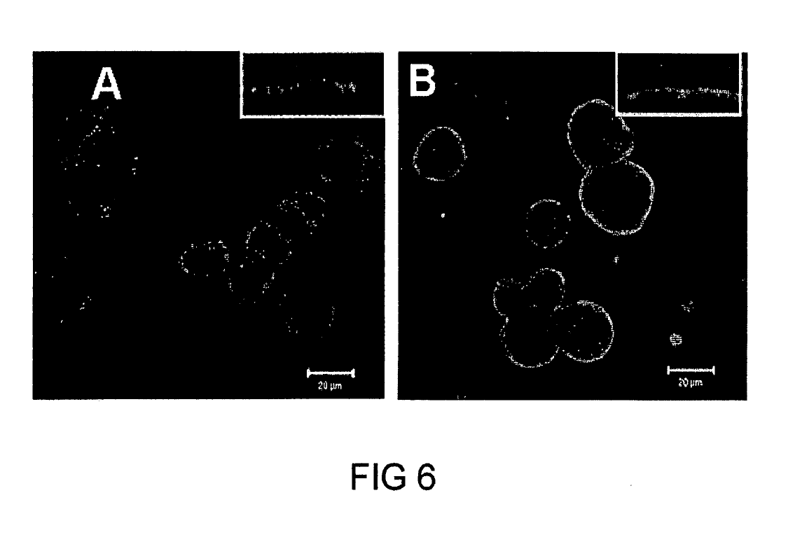

Recent studies established that ABCA1 resides both on the plasma membrane and in endocytic vesicles (Neufeld, E. B. et al. J. Biol. Chem., 2001, 276, 27584), and demonstrated the role of endosomal ABCA1 in the apoA-I-mediated efflux of cellular lipids (Neufeld, E. B. et al. J. Biol. Chem., 2004, 279, 15571). Thus, not only abundance of ABCA1, but also its localization are important for its function. Localization of ABCA1 in RAW macrophages transfected with viral protein Nef was therefore examined using confocal microscopy. A dramatic re-distribution of ABCA1 in the cells was observed (FIG. 6). Consistent with the findings of Neufeld et al. in cells transfected with an empty vector ABCA1 was distributed evenly between cytoplasm and plasma membrane, whereas transfection with Nef-expressing construct resulted in concentration of ABCA1 at the plasma membrane. A similar re-distribution of ABCA1 to the plasma membrane associated with inhibition of cholesterol efflux from RAW 254.7 macrophages after treatment with cyclosporine A was recently described by Le Goff et al. (Le Goff, W. et al. Arterioscler. Thromb. Vasc. Biol., 2004, 24, 2155). This finding demonstrates the ability of antibodies to detect re-localization of ABCA1 in the cultured cells. Nef-mediated redistribution of ABCA1 may contribute into impairment of cholesterol efflux impairment observed in Nef-transfected RAW cells and in HIV-infected macrophages.

### Example 8

### ApoA-I Binding, Internalization and Degradation

To test if inhibition or stimulation of cholesterol efflux results from changes in apoA-I binding to cells, the effect of the antibodies on interaction of ¹²⁵I-apoA-I with activated THP-1 cells was studied. The antibody NDF3F9, which had a limited effect on cholesterol efflux (FIG. 10), inhibited ¹²⁵I-apoA-I binding and internalization by TO-901317-activated THP-1 cells (FIGS. 12A, 12B). The antibody NDF4C2, that inhibited cholesterol efflux (FIG. 10), had a limited effect on ¹²⁵I-apoA-I binding and internalization by the cells (FIGS. 12A, 12B). The antibody NDF6F1, which inhibited or activated cholesterol efflux depending on whether or not ABCA1 expression was activated (FIG. 11), inhibited ¹²⁵I-apoA-I binding to both activated and non-activated cells (FIG. 12A). However, the effect of NDF6F1 on ¹²⁵I-apoA-I internalization was limited (FIG. 12B). Degradation of ¹²⁵I-apoA-I was negligible and was not affected by the antibodies (not shown). These findings suggest that blocking of apoA-I binding to ABCA1 is neither necessary nor sufficient for the inhibition of cholesterol efflux.

### Example 9

### ABCA1 Stability

Applicants investigated whether the antibody NDF6F1, which competes with apoA-I for binding (FIG. 6A), affects ABCA1 stability in similar manner to the effect of apoA-I. ABCA1 expression in THP-1 cells was boosted by incubation for 18 h with TO-901317 (4 μmol/L), cells were washed and then incubated for 18 h in serum-free medium alone or medium containing apoA-I (30 μg/ml), antibody NDF6F1 (5 μg/ml) or co-incubated with the NDF6F1 hybridoma cells (as a permanent source of the antibody). Most of the cellular ABCA1 disappeared when cells were incubated with the medium alone, but a significant proportion of ABCA1 was preserved when cells were incubated in the presence of apoA-I or NDF6F1 and especially when co-cultured with the hybridoma cells (FIG. 7A). The antibody NDF4C2 tested in a similar experiment did not protect ABCA1 from degradation (not shown). A similar experiment was conducted to test the effect of the antibodies specifically on the cell surface abundance of ABCA1 using biotinylation technique. THP-1 cells were incubated for 24 h with TO-901317 (4 μmol/L), washed and incubated for 18 h in the serum-free medium containing TO-901317 (4 μmol/L), antibodies NDF6F1 or NDF4C2 (10 μg/ml) or apoA-I (50 μg/ml). Cells were then biotinylated, membrane fraction isolated and biotinylated proteins precipitated by immobilized streptavidin and analyzed by Western blotting. Considerably more ABCA1 was detected at the cell surface when cells were incubated with TO-901317, the antibody NDF6F1 or apoA-I compared to the medium alone or the antibody NDF4C2 (FIG. 7B).

To further confirm the effect of the NDF6F1 antibody on ABCA1 stability, a time course of ABCA1 degradation was investigated using in-cell Western blot (FIG. 8). Expression of ABCA1 in THP-1 cells was boosted by TO-901317; the LXR agonist was then removed and cells incubated for various periods of time with serum-free medium alone (control), fresh TO-901317 (4 μmol/L), apoA-I (50 μg/ml) or NDF6F1 or NDF4C2 antibodies (10 μg/ml). Continuous incubation of cells with TO-901317 lead to an additional 30% increase in ABCA1 abundance over 24 h; addition of NDF6F1 instead of TO-901317 had almost the same effect on ABCA1 abundance. In contrast, incubation in the absence TO-901317 or in the presence of NDF4C2 antibody lead to a time-dependent decrease in ABCA1 by 40-60%. Incubation with apoA-I preserved ABCA1 at the initial level (FIG. 8). Thus the antibody NDF6F1, but not NDF4C2, can mimic apoA-I in stabilizing ABCA1 at the cell surface, which may be a mechanism for stimulating cholesterol efflux by this antibody.

To investigate the contribution of ABCA1 stabilization to the NDF6F1 effect on cholesterol efflux, HeLa cells (known not to have ABC transporters) were transiently transfected with ABCA1 or an ABCA1 mutant lacking the PEST domain, the site on ABCA1 responsible for its degradation by calpain proteases (Wang, N., Chen, W., Linsel-Nitschke, P., Martinez, L. O., Agerholm-Larsen, B., Silver, D. L., and Tall, A. R. (2003) J. Clin. Invest. 111, 99-107). As expected, transfection of HeLa cells with ABCA1 stimulated cholesterol efflux (FIGS. 9A, 9B). Similar to observations with macrophages, preincubation of HeLa-ABCA1 cells with NDF6F1 antibody stimulated cholesterol efflux, while preincubation with NDF4C2 antibody inhibited it (FIG. 9B). Consistent with the findings of Chen et al. (Chen, W., Wang, N., and Tall, A. R. (2005) J. Biol. Chem. 280, 29277-29281), transfection of HeLa cells with ΔPEST-ABCA1 increased cholesterol efflux compared to cells transfected with ABCA1 (p<0.01) raising it to the level observed in cells preincubated with NDF6F1. Preincubation of HeLa cells transfected with ΔPEST-ABCA1 with the antibody NDF6F1 did not further increase cholesterol efflux, while antibody NDF4C2 still inhibited efflux (FIGS. 9B, 9C). Preincubation of mock-transfected cells with NDF6F1 or NDF4C2 did not affect cholesterol efflux (FIG. 9A). Thus, preincubation of cells with the antibody NDF6F1 and the deletion of PEST domain had similar and non-additive effects on cholesterol efflux, indicating that the mechanism involved may be similar, e.g., inhibition of ABCA1 degradation.

### Example 10

### Intracellular Cholesterol Content

To assess the effect of the antibodies on intracellular cholesterol content, cholesterol and cholesteryl ester synthesis in RAW 264.7 macrophages were measured in the presence of the antibodies (final concentration 5 μg/ml) and apoA-I. RAW 264.7 macrophages were used in these experiments because they are more susceptible than THP-1 cells to cholesterol loading using acetylated LDL (acLDL). Increase of intracellular cholesterol content was expected to trigger compensatory suppression of cholesterol biosynthesis (Brown, M. S., and Goldstein, J. L. (1999) Proc Natl Acad Sci USA 96, 11041-11048) and a higher rate of cholesteryl ester synthesis (Chirinos, J. A., Zambrano, J. P., Chakko, S., Schob, A., Goldberg, R. B., Perez, G., and Mendez, A. J. (2005) Circulation 112, 2446-2453); the opposite was expected if cell cholesterol content decreased. Consistent with this view, cholesterol loading of cells with acLDL resulted in a significantly lower rate of cholesterol biosynthesis and significantly higher rate of cholesteryl ester formation (FIGS. 16A, 16B). Preincubation of cells with the antibody NDF6F1 resulted in a higher rate of cholesterol biosynthesis (FIG. 16A) and a lower rate of cholesteryl ester formation (FIG. 16B) in both cholesterol-loaded and not cholesterol loaded cells. This is consistent with lower intracellular cholesterol content most likely due to enhanced cholesterol efflux. Pre-incubation of cells with the antibody NDF4C2 resulted in a lower rate of cholesterol biosynthesis and higher rate of cholesteryl ester formation whether or not cells were cholesterol-loaded (FIGS. 16A, 16B) being consistent with higher intracellular cholesterol content due to inhibition of cholesterol efflux.

The foregoing is intended as illustrative of the present invention but is not limiting. Numerous variations and modifications can be effected without departing from the spirit and scope of the novel concepts of the invention. It is to be understood that no limitations with respect to the specific antibodies, compositions and uses described herein is intended or should be inferred.

1. A binding domain of ABCA1, wherein binding of a ligand to the domain is capable of modulating a biological activity of ABCA1.

2. A binding domain according to claim 1, wherein the biological activity is selected from the group consisting of: apoA-binding, internalization, localization, stability, ability to efflux cholesterol, and ability to efflux phospholipid.

3. A binding domain according to claim 1 including or consisting of a first and/or second region of the ABCA1.

4. A binding domain according to claim 1 wherein the first region includes or consists of the extracellular loop connecting the first and second transmembrane domains of ABCA1.

5. A binding domain according to claim 3 wherein the first region includes or consists of amino acid residues from the human ABCA1 region from about position 602 to about position 620, or a functional equivalent or fragment thereof.

6. A binding domain according to claim 3 wherein the first region includes or consists of amino acid residues from the mouse ABCA1 region from position 542 to about position 560 or a functional equivalent or fragment thereof.

7. A binding domain according to claim 3 wherein the first region includes or consists of the sequence:
(SEQ ID NO:1)
CAIIRVLTGTEKKTGVYMQ
or a functional equivalent or fragment thereof.

8. A binding domain according to claim 3 wherein the second region includes or consists of part of the extracellular loop connecting the 7^{th} and 8^{th} transmembrane domains of ABCA1.

9. A binding domain according to claim 3 wherein the second region includes or consists of amino acid residues from the mouse ABCA1 region from about position 1251 to about position 1390 or a functional equivalent or fragment thereof.

10. A binding domain according to claim 3 wherein the second region includes or consists of amino acid residues from the human ABCA1 region from about position 1311 to about position 1450 or a functional equivalent or fragment thereof.

11. A binding domain according to claim 3 wherein the second region includes or consists of the sequence:
(SEQ ID NO:2)
MDGKGSYQLKGWKLTQQQFVALLWKRLLIARRSRKGFFAQIVLPAVFVCI
ALVFSLIVPPFGKYPSLELQPWMYNEQYTFVSNDAPEDMGTQELLNALTK
DPGFGTRCME GNPIPDTPCLAGEEDWTISPVPQSIVDLFQ.
or a functional equivalent or fragment thereof.

12. An antibody capable of binding to a binding domain according to claim 1.

13. An antibody according to claim 12 selected from the group consisting of NDF4C2, NDF3F9, NDF2D12, and NDF6F1 or a functional equivalent or fragment thereof.

14. An antibody according to claim 13 wherein where antibody is NDF4C2 or NDF2D12 the antibody is IgM.

15. An antibody according to claim 13 wherein where the antibody is NDF3F9 the antibody is IgG₃.

16. An antibody according to claim 12 obtained directly or indirectly from a hybridoma cell deposited with the Korean Cell Line Research Foundation according to the following accession details:

| | |
|---|---|
| NDF4C2 | KCLRF-BP-00094 |
| NDF3F9 | KCLRF-BP-00095 |
| NDF2D12 | KCLRF-BP-00097 |
| NDF6F1 | KCLRF-BP-00096 |

17. A method of identifying an ABCA1 binding domain agonist or antagonist the method including exposing a potential agonist or antagonist to an ABCA1 binding domain or portion thereof according to claim 1, and determining the ability of the potential agonist or antagonist to bind to the ABCA1 binding domain or otherwise interfere with the binding of the ABCA1 binding domain with an associated molecule.

18. A method according to claim 17 wherein the associated molecule is apoA-I.

19. A method according to claim 17 wherein the ability of the potential agonist or antagonist to bind to the ABCA1 binding domain or otherwise interfere with the binding of the ABCA1 binding domain with another involves the use of an antibody that specifically binds to a ligand-binding domain of ABCA1.

20. An ABCA1 agonist or antagonist identified by a method according to claim 17.

21. A method for treating or preventing a disease related to a biological activity of ABCA1, the method including administering to a subject in need thereof an effective amount of an antibody according to claim 12 or an ABCA1 agonist or antagonist.

22. A method according to claim 21 wherein the biological activity is selected from the group consisting of: apoA-binding, internalization, localization, stability, ability to efflux cholesterol, and ability to efflux phospholipid.

23. A method according to claim 21 wherein the disease related to ABCA1 activity is a disease related to cholesterol efflux or phospholipid efflux.

24. A method according to claim 21 wherein the disease is selected from the group consisting of Tangier disease, coronary heart disease, atherosclerosis, and acquired immune deficiency syndrome.

25. A method according to claim 21 wherein the subject is a human or a laboratory test animal.

26. A method according to claim 21 wherein the subject is a human.

27. A method of modulating a biological activity of ABCA1, the method including exposing ABCA1 to an antibody according to claim 12, or an ABCA1 agonist or antagonist.

28. A method according to claim 27 wherein the biological activity is selected from the group consisting of: apoA-binding, internalization, localization, stability, ability to efflux cholesterol, and ability to efflux phospholipid.

29. A method for modulating cholesterol efflux or phospholipid efflux in a cell the method including exposing ABCA1 in the cell to an antibody according to claim 12 or an ABCA1 agonist or antagonist.

30. A method of modulating the level of HDL in the blood of an animal the method including exposing the animal to an antibody according to claim 12 or an ABCA1 agonist or antagonist.

31. A method of immunoassay for determining blood, serum, plasma or tissue levels of ABCA1 including use of an antibody according to claim 12.

32. A method of monitoring a patient having, or at risk of having, a vascular disorder or a disorder related to an HDL receptor including use of an antibody according to claim 12.

33. A method of isolating a subpopulation of cells from a population of cells, the method including exposing the population of cells to an antibody according to claim 12 and separating the cells that specifically bind to the antibody from those that do not specifically bind to the antibody.

34. A subpopulation of cells isolated using a method according to claim 33.
